(19) United States
(12) Patent Application Publication
(10) Pub. No.: US 2011/0313028 A1
(43) Pub. Date: Dec. 22, 2011

(54) SUPPRESSOR OF AP-1

(75) Inventors: Paul B. Fisher, Richmond, VA (US); Zao-zhong Su, Richmond, VA (US); Seok-Geun Lee, Richmond, VA (US); Devanand Sarkar, Richmond, VA (US)

(73) Assignee: VIRGINIA COMMONWEALTH UNIVERSITY, Richmond, VA (US)

(21) Appl. No.: 13/130,745

(22) PCT Filed: Nov. 25, 2009
(86) PCT No.: PCT/US09/65958

§ 371 Date: Sep. 12, 2011

Related U.S. Application Data
(60) Provisional application No. 61/118,200, filed on Nov. 26, 2008.

(51) Int. Cl. A61K 48/00; A61P 35/00; C12N 15/85; C12N 5/10; C07H 21/04; C12N 15/63

(52) U.S. Cl. 514/44 R; 536/23.5; 435/320.1; 435/455; 435/325

(57) ABSTRACT

Methods, compositions, and therapeutic products for use in the field of oncology and specifically, for the treatment of cancer and other diseases in which SARI is ameliorative or therapeutic and/or small molecule screening for anti-cancer drugs are provided. Cancer specific gene expression using the CCN1 promoter, which is negatively regulated by SARI, for targeting therapeutic molecules in tumors is also provided.

### CROSS-REFERENCE TO RELATED APPLICATIONS

This application claims priority to and the benefit under 35 U.S.C. §119(e) to U.S. Provisional Appln. Ser. No. 61/118,200, filed Nov. 26, 2008, the disclosure of which is expressly incorporated by reference herein in its entirety.

### STATEMENT AS TO RIGHTS TO INVENTIONS MADE UNDER FEDERALLY SPONSORED RESEARCH AND DEVELOPMENT

This invention was made, at least in part, with U.S. government support under National Institutes of Health Grants R01 CA035675, R01 CA097318 and P01 CA104177. The U.S. government has certain rights in the invention.

### BACKGROUND OF THE INVENTION

Although interferons (IFNs) were originally identified as proteins conferring viral interference, over the last fifty years a variety of effects have been attributed to these molecules, such as inhibition of viral, bacterial and parasitic pathogenesis, inhibition of cell growth, induction of apoptosis, inflammation, immunomodulation and anti-angiogenesis (Borden E C et al., Nat Rev Drug Discov, 6:975-990 (2007); Stark G R et al., Annu Rev Biochem, 67:227-264 (1998); Zhang S Y et al., Immunol Rev, 220:225-236 (2007); Takeuchi O & Akira S, Immunol Rev, 220:214-224 (2007); Barber G N, Semin Cancer Biol, 10:103-111 (2000); Fisher P B & Grant S, Pharmacol Ther, 27:143-166 (1985)). IFNs exert their diverse effects by stimulating the expression of a plethora of IFN-stimulated genes (ISGs) in target cells (Stark G R et al., Annu Rev Biochem, 67:227-264 (1998)). Both type I (IFN-α, IFN-β, IFN-ω and IFN-τ) and type II (IFN-γ) IFNs exert potent anti-tumor effects and are used clinically either as a monotherapy or as an adjuvant to chemotherapy or radiotherapy for a number of solid tumors and hematological malignancies that include melanoma, renal cell carcinoma, Kaposi's sarcoma, malignant glioma, lymphomas and leukemias (Borden E C et al., Nat Rev Drug Discov, 6:975-990 (2007)). The anti-tumor effects of IFNs are mediated by direct inhibition of proliferation of cancer cells as well as by indirect effects such as inhibition of tumor angiogenesis and upregulation of tumor-specific antigens and adhesion molecules.

In an in vitro system, treatment of human melanoma cells with type I IFN (IFN-β) and the protein kinase C activator mezerein (MEZ) induces irreversible growth arrest and ‘terminal differentiation’ (Fisher P B et al., Anticancer Res, 6:765-774 (1986); Fisher P B et al., J Interferon Res, 5:11-22 (1985)). Differential gene expression analysis between IFN-β+MEZ-treated terminally differentiated versus control melanoma cells identified a variety of novel ‘melanoma differentiation associated (mda)’ genes the functional characterization of which has revealed their central role in various IFN-regulated events, such as viral interference, growth inhibition and induction of apoptosis (Jiang H, Kang D C, Proc Natl Acad Sci USA, 97(23):12684-12689 (2000); Jiang H et al., Mol Cell Differ, 2:221-239 (1994); Jiang H et al. Oncogene, 10:1855-1864 (1995); Jiang H et al., Oncogene, 11:1179-1189 (1995); Jiang H et al., Oncogene, 11:2477-2486 (1995); Jiang H et al., Mol Cell Differ, 1:41-66 (1993); Kang D C et al., Proc Natl Acad Sci USA, 99:637-642 (2002); Kang D C et al., Proc Natl Acad Sci USA, 95:13788-13793 (1998)).

Activator protein-1 (AP-1), one of the first identified mammalian transcription factors, plays an essential role in regulating cell proliferation and oncogenic transformation (Angel P & Karin M, Biochim Biophys Acta, 1072:129-157 (1991)). AP-1 comprises a variety of dimeric basic region-leucine zipper (bZIP) proteins that belong to the Jun (c-Jun, JunB, JunD), Fos (c-Fos, FosB, Fra-1 and Fra-2), Maf and ATF sub-families that recognize either the 12-O-tetradecanoylphorbol-13-acetate (TPA) response element (TRE, 5′-TGAG/CTCA-3) or the cAMP response element (CRE, 5′-TGACGTCA-3′) (Chinenov Y & Kerppola T K, Oncogenem, 20:2438-2452 (2001)). Among the Jun proteins, c-Jun is the most potent transcriptional activator and the c-Fos-c-Jun heterodimer positively regulates cell proliferation and transformation (Angel P & Karin M, Biochim Biophys Acta, 1072:129-157 (1991); Maki Y et al., Proc Natl Acad Sci USA, 84:2848-2852 (1987); Ryseck R P & Bravo R, Oncogene, 6:533-542 (1991)). c-Jun co-operates with Ha-ras in rat embryonic fibroblast transformation and c-jun-l-cells are refractory to the transforming activity of oncogenic Ras (Vandel L et al., Mol Cell Biol, 16:1881-1888 (1996); Johnson R et al., Mol Cell Biol, 16:4504-4511 (1996)). Additionally, overexpression of c-Jun itself can immortalize rodent fibroblasts in culture Vandel L et al., Mol Cell Biol, 16:1881-1888 (1996)). Augmented AP-1 activity has been observed in more than 90% of human cancers indicating its seminal importance in human carcinogenesis Eferl R & Wagner E F, Nat Rev Cancer, 3:859-868 (2003)).

### BRIEF SUMMARY OF THE INVENTION

This invention provides methods compositions, and therapeutic products for the treatment of cancer and other diseases in which SARI is ameliorative or therapeutic and/or small molecule screening for anti-cancer drugs. The invention can be implemented in a number of ways.

An aspect of the invention includes an isolated nucleic acid that comprises a polynucleotide encoding a polypeptide substantially identical to (including but not limited to at least 95% identical to) SEQ ID NO:2. In some embodiments, the polypeptide comprises SEQ ID NO:2. In some embodiments, the polynucleotide comprises SEQ ID NO:1.

Another aspect of the invention may include an expression cassette having a promoter operably linked to a polynucleotide encoding a polypeptide substantially identical to (including but not limited to at least 95% identical to) SEQ ID NO:2. In some embodiments, a vector including an expression cassette having a promoter operably linked to a polynucleotide encoding a polypeptide substantially identical to (including but not limited to at least 95% identical to) SEQ ID NO:2 is provided. In some embodiments, a cell comprising a vector as described herein (i.e., comprising an expression cassette as described herein) is provided. The expression cassette may be heterologous to the cell.

A further aspect of the invention is an isolated antisense oligonucleotide or small interfering RNA (siRNA) comprising or complementary to all or a portion of a messenger RNA (mRNA) having SEQ ID NO:3 and encoding a SARI protein, where the antisense oligonucleotide or small interfering RNA (siRNA) inhibits production of the SARI protein. In some embodiments, the expression cassette comprises a promoter operably linked to a polynucleotide comprising the antisense oligonucleotide or small interfering RNA (siRNA) having SEQ ID NO:3 and encoding a SARI protein, where the antisense oligonucleotide or small interfering RNA (siRNA) inhibits production of the SARI protein. In some embodiments, the expression cassette comprises a promoter operably linked to a polynucleotide comprising the antisense oligonucleotide or small interfering RNA (siRNA) having SEQ ID NO:3 and encoding a SARI protein, where the antisense oligonucleotide or small interfering RNA (siRNA) inhibits production of the SARI protein. A vector may include an expression cassette that includes a promoter operably linked to a polynucleotide comprising the antisense oligonucleotide or small interfering RNA (siRNA) having SEQ ID NO:3 and encoding a SARI protein, where the antisense oligonucleotide or small interfering RNA (siRNA) inhibits production of the SARI protein. A cell may include the expression cassette or expression vector that includes a promoter operably linked to a polynucleotide comprising the antisense oligonucleotide or small interfering RNA (siRNA) having SEQ ID NO:3 and encoding a SARI protein, where the antisense oligonucleotide or small interfering RNA (siRNA) inhibits production of the SARI protein and the expression cassette or expression vector may be heterologous to the cell.

An aspect of the invention may include an isolated polypeptide comprising an amino acid sequence substantially identical to (including but not limited to at least 95% identical to) SEQ ID NO:2. The isolated polypeptide may comprise SEQ ID NO:2.

Another aspect of the invention includes a method of suppressing cancer cell growth and/or inducing apoptosis in a cancer cell by introducing an expression cassette having a promoter operably linked to a polynucleotide encoding a polypeptide substantially identical to (including but not limited to at least 95% identical to) SEQ ID NO:2 into the cell and inducing expression of the polypeptide encoded by the expression cassette. A vector may include the expression cassette having a promoter operably linked to a polynucleotide encoding a polypeptide substantially identical to (including but not limited to at least 95% identical to) SEQ ID NO:2 into the cell and inducing expression of the polypeptide encoded by the expression cassette. The vector may be a viral vector. The viral vector may be an adenoviral vector. A cell may include a vector having an expression cassette having a promoter operably linked to a polynucleotide encoding a polypeptide substantially identical to (including but not limited to at least 95% identical to) SEQ ID NO:2 into the cell and inducing expression of the polypeptide encoded by the expression cassette.

The method may be performed in vitro and/or in vivo. The cancer cell may express CCN1 or CCN2 before the induced expression of the polypeptide. The cancer cell may express AEG-1 before the induced expression of the polypeptide. The cancer cell may include a breast cancer cell, a glioblastoma multiforme cell, a lung cancer cell, a melanoma cell, a prostate cancer cell, a ovarian cancer cell, a cervical cancer cell, an osteosarcoma cell, a fibrosarcoma cell, and a liver cancer cell.

Another aspect of the invention includes an isolated nucleic acid including a promoter polynucleotide substantially identical to (including but not limited to at least 95% identical to) SEQ ID NO:4, where the promoter polynucleotide is Protein Kinase C-inducible. The promoter polynucleotide may include SEQ ID NO:4. The promoter may be operably linked to a heterologous polynucleotide. The heterologous polynucleotide may encode a polypeptide. The polypeptide may be a toxin, a protein that induces apoptosis, and/or a reporter protein. The heterologous polynucleotide may encode an antisense oligonucleotide or an siRNA.

A further aspect of the invention includes an isolated nucleic acid comprising a promoter polynucleotide substantially identical to (including but not limited to at least 95% identical to) SEQ ID NO:5, wherein the promoter polynucleotide initiates cancer-specific gene expression. The promoter polynucleotide may comprise SEQ ID NO:5.

A yet further aspect of the invention includes a method of inducing expression of a polynucleotide of interest in a cell by introducing an expression cassette into the cell, the expression cassette comprising a SARI promoter polynucleotide operably linked to a polynucleotide of interest, where the promoter polynucleotide is substantially identical to (including but not limited to at least 95% identical to) SEQ ID NO:4 and initiates cancer-specific gene expression, and contacting the cell with an inducer of SARI promoter expression, thereby inducing expression of the polynucleotide of interest. The inducer is interferon or a protein kinase C activator. The protein kinase C activator is mezerin.

Another aspect of the invention includes an isolated nucleic acid comprising an expression cassette comprising a CCN1 promoter operably linked to a polynucleotide encoding mda-7/IL-24. An expression cassette may include an isolated nucleic acid comprising an expression cassette comprising a CCN1 promoter operably linked to a polynucleotide encoding mda-7/IL-24. A vector may include an expression cassette having an isolated nucleic acid comprising an expression cassette comprising a CCN1 promoter operably linked to a polynucleotide encoding mda-7/IL-24. The vector may be a viral vector. The viral vector may be an adenoviral vector.

An aspect of the invention also include an antibody that binds to a polypeptide consisting of SEQ ID NO2.

Additional features, advantages, and embodiments of the invention may be set forth or apparent from consideration of the following detailed description, and claims. Moreover, it is to be understood that both the foregoing summary of the invention and the following detailed description are exemplary and intended to provide further explanation without limiting the scope of the invention as claimed.

### Definitions

Provided immediately below is a “Definition” section, where certain terms related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined by, e.g., Kabat et al. (Kabat et al., Sequences of Proteins of Immunological Interest, Fifth Edition, U.S. Department of Health and Human Services, U.S. Government Printing Office (1991)).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see FUNDAMENTAL IMMUNOLOGY (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies refer to antibodies derived from a single clone. Techniques for the production of single chain antibodies (U.S. Pat. No. 4,946,778) can be adapted to produce antibodies to polypeptides. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., Proc. Natl. Acad. Sci. USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. Embodiments provide polypeptides or polynucleotides that are substantially identical to the polypeptides or polynucleotides, respectively, exemplified herein (e.g., the SARI polypeptides and polynucleotides presented herein, as well as the SARI and CCN1 promoters provided herein). Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The terms “short-inhibitory RNA” and “siRNA” interchangeably refer to short double-stranded RNA oligonucleotides that mediate RNA interference (also referred to as “RNA-mediated interference,” or RNAi). RNAi is a highly conserved gene silencing event functioning through targeted destruction of individual mRNA by a homologous double-stranded small interfering RNA (siRNA) (Fire, A. et al., Nature 391:806-811 (1998)). Mechanisms for RNAi are reviewed, for example, in Bayne and Allshire, Trends in Genetics (2005) 21:370-73; Morris, Cell Mol Life Sci (2005) 62:3057-66; Filipowicz, et al., Current Opinion in Structural Biology (2005) 15:331-41.

The term “fragments or “fragments thereof,” as used herein, generally refers to proteins, or nucleic acids which encode a polypeptide or a polypeptide of at least about 25 amino acid residues, e.g., at least about 50, at least about 75, at least about 100, or greater than about 100 amino acid residues.

The term “gene,” as used herein, generally refers to a DNA sequence that comprises control and coding sequences necessary for the production of a polypeptide or precursor thereof. The polypeptide can be encoded by a full length coding sequence or by any portion of the coding sequence so long as the desired enzymatic activity is retained. The term “gene” encompasses both cDNA and genomic forms of a given gene.

An “antisense gene,” as used herein, may be constructed by reversing the orientation of the gene with respect to its promoter so that the antisense strand is transcribed.

An “antisense RNA,” as used herein, generally refers to an RNA molecule complementary to a particular RNA transcript that can hybridize to the transcript and block its function.

The terms “complementary” or “complementarity,” as used herein, include the natural binding of polynucleotides permissive conditions by base-pairing. For example, the sequence “A-G-T” binds to the complementary sequence “T-C-A.” Complementarity between two single-stranded molecules may be “partial,” in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in antisense or nonsense nucleic acid reactions, such as antisense RNA, which depend upon binding between nucleic acids strands and in the design and use of molecules.

The term “deletion,” as used herein, generally refers to a change in the amino acid or nucleotide sequence and results in the absence of one or more amino acid residues or nucleotides.

The term “insertion” or “addition,” as used herein, generally includes a change in an amino acid or nucleotide sequence resulting in the addition of one or more amino acid residues or nucleotides, respectively, as compared to the naturally occurring molecule.

The term “introduction,” as used herein generally refers to insertion of a nucleic acid sequence into a cell, by methods including infection, transfection, transformation or transduction.

An “expression cassette,” as used herein generally refers to a combination of regulatory elements that are required by the host for the correct transcription and translation (expression) of the genetic information contained in the expression cassette. These regulatory elements comprise a suitable (i.e., functional in the selected host) transcription promoter and a suitable transcription termination sequence.

The term nucleic acid “control sequences,” as used herein, generally refers collectively to promoter sequences, polyadenylation signals, transcription termination sequences, upstream regulatory domains, enhancers, and the like, untranslated regions, including 5′-UTRs and 3′-UTRs, which collectively provide for the transcription and translation of a coding sequence in a host cell.

The term “operably linked,” as used herein, generally refers to an arrangement of nucleotide sequence elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. A control sequence “directs the transcription” of a coding sequence in a cell when RNA polymerase will bind the promoter sequence and transcribe the coding sequence into mRNA, which is then translated into the polypeptide encoded by the coding sequence.

“Promoter,” as used herein generally includes a regulatory region of DNA capable of initiating, directing and mediating the transcription of a nucleic acid sequence. Promoters may additionally include recognition sequences, such as upstream or downstream promoter elements, which may influence the transcription rate.

“Inducible promoter,” as used herein generally refers to a promoter where the rate of RNA polymerase binding and initiation is modulated by external stimuli. Such stimuli include light, heat, anaerobic stress, alteration in nutrient conditions, presence or absence of a metabolite, presence of a ligand, microbial attack, wounding and the like.

“Viral promoter,” as used herein generally refers to a promoter with a DNA sequence substantially similar to the promoter found at the 5′ end of a viral gene. A typical viral promoter is found at the 5′ end of the gene coding for the p2I protein of MMTV.

“Synthetic promoter,” as used herein generally refers to a promoter that was chemically synthesized rather than biologically derived. Usually synthetic promoters incorporate sequence changes that optimize the efficiency of RNA polymerase initiation.

“Constitutive promoter,” as used herein generally refers to a promoter where the rate of RNA polymerase binding and initiation is approximately constant and relatively independent of external stimuli.

The term “coding region” or a “nucleotide sequence encoding” a particular protein, is a nucleic acid sequence which is transcribed and translated into a polypeptide in vivo or in vitro when placed under the control of appropriate regulatory sequences. The boundaries of the coding sequence are determined by a start codon at the 5′-(amino) terminus and a translation stop codon at the 3′-(carboxy) terminus. A coding sequence can include, but is not limited to, procaryotic sequences, cDNA from eucaryotic mRNA, genomic DNA sequences from eucaryotic (e.g., mammalian) sources, viral RNA or DNA, and even synthetic nucleotide sequences. A transcription termination sequence will usually be located 3′ to the coding sequence.

“Inhibition,” as used herein, generally refers to a reduction in the parameter being measured. The amount of such reduction is measured relative to a standard (control). The preferred detection products may include newly transcribed mRNA and a DNA-DNABP complex. “Reduction” is defined herein as a decrease of at least about 25% relative to control, e.g., at least about 50%, or at least about 75%.

“Transfection,” as used herein includes the process of introducing a DNA expression vector into a cell. Various methods of transfection are possible including microinjection or lipofection.

“Transformation” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, and lipofection.

The term “host cell,” as used herein generally refers to any prokaryotic or eukaryotic cell such as bacteria cells, mammalian cells, fungal cells, yeast cells, and insect cells.

“Functional equivalent,” as used herein, generally refers to a protein or nucleic acid molecule that possesses functional or structural characteristics that is substantially similar to a protein, polypeptide, enzyme, or nucleic acid. A functional equivalent of a protein may contain modifications depending on the necessity of such modifications for the performance of a specific function. The term “functional equivalent” is intended to include the “fragments,” “mutants,” “hybrids,” “variants,” “analogs,” or “chemical derivatives” of a molecule.

The term “purification,” as used herein, generally refers to any process by which proteins, polypeptides, or nucleic acids are separated from other elements or compounds on the basis of charge, molecular size, or binding affinity.

The phrase “substantially purified,” or “substantially isolated,” as used herein generally includes nucleic or amino acid sequences that are removed from their natural environment, isolated or separated, and are at least about 60% free, specifically at least about 75% free, and most specifically at least about 90% free from other components with which they may be associated with, and includes recombinant or cloned nucleic acid isolates and chemically synthesized analogs or analogs biologically synthesized by systems.

“Vector,” as used herein generally refers to a cloning vector that is designed so that a coding nucleic acid sequence inserted at a particular site will be transcribed and translated. A typical expression vector may contain a promoter, selection marker, nucleic acids encoding signal sequences, and regulatory sequences, e.g., polyadenylation sites, 5′-untranslated regions, and 3′-untranslated regions, termination sites, and enhancers. “Vectors” may include viral derived vectors, bacterial derived vectors, plant derived vectors, and insect derived vectors.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced.

FIG. 1 shows the genomic structure of the SARI gene. The white boxes represent the exons and the black lines represent the introns. The numbers at the bottom of the white boxes represent the size of the exons and those on top of the black lines represent the size of the introns in bp. The arrow indicates the translation start site and the octagon indicates the stop codon.

FIG. 2 shows the amino acid sequence of the SARI protein. The leucine residues in the leucine zipper are underlined.

FIG. 3 shows quantitative SARI mRNA expression analysis in cancer (T98G, DU145, and HO-1) and normal cells (PHFA, P69, and FM516-SV; in arbitrary units [a.u.]). Data represent mean±SD of three independent experiments.

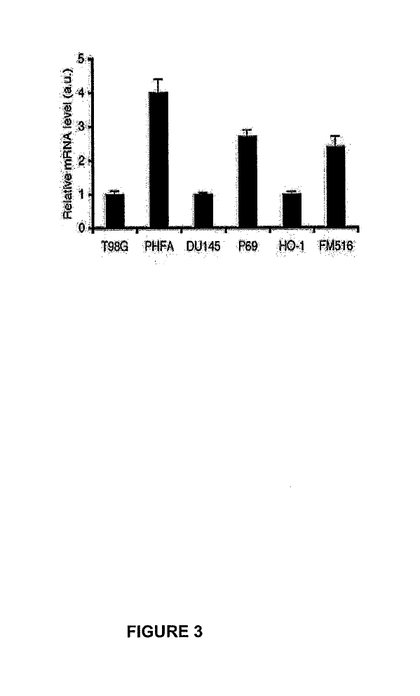

FIG. 4 illustrates that SARI suppresses growth of cancer cells in vitro and in vivo. (Panel A) Cancer (H4, DU145, and MeWo) and normal cells (PHFA, P69, and FM-516SV) were infected with either Ad.vec or Ad.SARI at a multiplicity of infection of 100 pfu/cell and cell number was counted at the indicated time points. The data represent mean±SD of three independent experiments. (Panel B) Cancer (H4, DU145, and HeLa) and normal cells (PHFA) were infected as in A and apoptosis was analyzed by Annexin V binding assay (Upper) and propidium iodide staining assay (Lower), followed by flow cytometry 48 h after infection. (Panel C) Subcutaneous xenografts were established in the flanks of athymic nude mice with DU145 human prostate cancer cells. Established tumors were injected with PBS, Ad.vec, or Ad.SARI as described in Materials and Methods and survival of animals was analyzed by the Kaplan-Meier test.

FIG. 5 illustrates that SARI inhibits growth of transformed cells in a Ras- and Src-dependent manner. (Panel A) Normal immortal rat embryonic fibroblasts (CREF) and clones of CREF transformed by H-ras, v-src, HPV-18, or a specific temperature-sensitive mutant of type 5 adenovirus H5hrl (CREF-ras, CREF-src, CREF-HPV, and CREFH5 hr1, respectively) were infected with either Ad.vec or Ad.SARI at a multiplicity of infection of 100 pfu/cell and cell number was counted at the indicated time points. (Panel B) Soft agar colony formation assay of CREF, CREF-ras, CREF-src, CREF-HPV, and CREF-H5hrl cells infected as in A. The data represent mean±SD of three independent experiments.

FIG. 6 illustrates that SARI inhibits AP-1 activation. CREF and CREF clones stably overexpressing c-jun (CREF-c-jun 13, CREF-c-jun 16, and CREF-c-jun 53) were treated and analyzed as in FIG. 5, Panel A.

FIG. 7 shows HeLa cells and HeLa cells stably expressing antisense SARI (HeLa-SARIAs) were treated with IFN-γ (100, 500, and 2,000 U/ml) and cell numbers were counted at the indicated time points.

FIG. 8 shows HeLa and HeLa-SARIAs cells that were transfected with AP-1-luc, treated with TPA or IFN-γ or a combination, and luciferase activity was monitored by a luminometer. Luciferase activity was normalized by β-galactosidase activity. Data represent mean±SD of three independent experiments.

FIG. 9 shows that SARI directly interacts with c-JUN. Mammalian two-hybrid assay was performed using pAD-c-jun andpBD-SARI in HeLa, DU145, FM516-SV, and MeWocells. Luciferase activity was normalized by β-galactosidase activity. The data represent mean±SD of three independent experiments.

FIG. 10 shows CREF-c-jun-13 cells transfected with empty vector (pcDNA) or expression plasmids expressing WT SARI or SARI-mt and subjected to clonogenic assay for 2 weeks upon selection with hygromycin. Data represent mean±SD of three independent experiments.

FIG. 11 shows that over-expression of SARI attenuates CCN2-induced anchorage-independent growth and invasion in various cancer lineages. The indicated cell types were transiently transfected with pcDNA3.1-CCN1 or control vector pcDNA3.1 (10 μg/10⁶ cells). After 24 h incubation, the transfected cells were infected with Ad.SARI (10 pfu/cell) or Ad.vec and 24 h after viral infection the cells were evaluated for anchorage-independent growth in soft agar. Colonies (>0.1-mm) were counted 2 wks after seeding.

FIG. 12 shows that SARI attenuates anchorage-independent growth and invasion in breast cancer cells stably over expressing CCN1. The indicated cells that were infected with Ad.SARI or Ad.vec at 10 pfu/cell for 24 h and after viral infection the cells were analyzed by soft agar cloning assays. Colonies (>0.1-mm) were counted 2 wks after seeding.

FIG. 13 shows that Ad.SARI eradicated nude mouse xenograft tumors generated by stable transfectants of MCF-7 that ectopically over-express CCN1. (Panel A) Tumor xenografts were established by injecting MCF-CCN1 cl1 and MCF-CCN1 cl2 (MCF-7 clones stably over expressing CCN1) in athymic nude mice, and tumors were injected with the indicated Ad over a 3 wk period (total of seven injections). A) Measurements of MCF-CCN1 cl1 and MCF-CCN1 cl2 xenograft tumor volumes. Points, average (with a minimum of five mice in each group); bars, +S.D. Inset: photograph of the animals of each representative group. (Panel B) Left, photograph of the MCF-CCN1 cl1 and MCF-CCN1 cl2 xenograft tumor at the end of the study; right, measurement of tumor weight at the end of the study. Columns, mean (with at least five mice in each group); bars, +S.D.C.) Tumors were isolated from MCF-CCN1 cl1 and MCF-CCN1 cl2 xenografts after seven injections with the indicated Ad. Formalin-fixed, paraffin-embedded sections were immunostained for Ki-67, SARI and CD31.

FIG. 14 shows that SARI reduced CCN1 promoter activity in different cancer lineages by inhibiting AP-1 activation. (Panel A) The indicated cells were transfected with wild type CCN1/pGL3Luc (10 μg/10⁶ cells) and after 24 h incubation, the transfected cells were infected with Ad.vec or Ad.SARI at 10 pfu/cell for another 24 h and luciferase assays were done. TPA (100 ng/ml) was added 8 h before luciferse assays. (Panel B) MDA-MB-231 cells were transfected with wild type CCN1/pGL3Luc or AP-1 mutant CCN1 (-AP-1)/pGL3Luc (CCN1/pGL3Luc promoter that lacks AP-1 binding site) at a concentration of 10 μg per 10⁶ cells. After 24 h incubation, the transfected cells were infected with Ad.vec or Ad.SARI at 10 pfu/cell for another 24 h and luciferase assays were done. TPA (100 ng/ml) was added 8 h before performing Luciferse assays.

FIG. 15 shows that SARI reduced CCN1 promoter function by inhibiting AP-1 activity in cloned rat embryo fibroblast cells (CREF) that stably express elevated c-Jun. (Panel A) Left panel: CREF and stable transfectants of CREF stably over-expressing c-Jun (CREF c-Jun-13 and CREF c-Jun-16) were transfected with wild type CCN1/pGL3Luc (10 μg/10⁶ cells). After 24 h incubation the transfected cells were infected with Ad.vec or Ad.SARI at 10 pfu/cell for another 24 h and Luciferase assays were done. Right panel: The indicated cells were infected with Ad.SARI or Ad.vec at 10 pfu/cell for 24 h and after viral infection the cells were seeded in soft agar to determine anchorage independent cloning efficiency. Colonies (>0.1-mm) were counted 2 wks after seeding. (Panel B) CREF c-Jun-13 and CREF c-Jun-16 cells were infected with Ad.SARI or Ad.vec at 10 pfu/cell for 24 h and after viral infection the cells were evaluated by Matrigel invasion assays. All experiments were performed at least three times, and data represent mean±S.D. (*P<0.05 vs MCF-pcDNA3.1+Ad.vec).

FIG. 16 illustrates a model for SARI inhibition of CCM-induced transformation progression by interfering with AP-1 activation. In this model it is demonstrated that SARI interacts with c-JUN and hence interferes with AP-1 activation of the CCN1 promoter causing down regulation of CCN1 expression. This phenomenon results in reduced integrin αvβ3 activation and consequently reduced CCN1-induced cell proliferation.

### DETAILED DESCRIPTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a cell” is a reference to one or more cells and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

### Introduction

This application discloses information related to the discovery of a new human gene product, SARI, the effect of SARI's on cancer cell survival, and SARI's regulation of the CCN1 promoter. Certain embodiments generally relate to methods, compositions, and therapeutic products (e.g., cell lines, monoclonal antibodies, polyclonal antibodies, polypeptide(s), protein(s), gene therapy vectors, microbubbles, nucleic acids, and transgenic knock-in or knock-out animals) for use in the field of oncology and specifically, for the treatment of cancer and other diseases in which SARI is ameliorative or therapeutic and/or small molecule screening for anti-cancer drugs. In particular, certain embodiments provide methods and therapeutic products for expressing or overexpressing the SARI gene product or a biologically active fragment thereof in cancer cells for use as an anti-cancer agent. Moreover, embodiments relate to therapeutic products for suppressing or inhibiting expression of the CCN1 gene product for use an anti-cancer agent.

Alternatively, other embodiments relate to suppressing or inhibiting expression of the SARI gene product as a means for screening for anti-cancer agents dependent upon SARI. Moreover, certain embodiments relate to methods and therapeutic products for expressing or overexpressing the expression of the CCN1 gene product for screening for anti-cancer agents. Other embodiments relate to therapeutic products for detecting the presence, absence, or expression level of the SARI gene product and/or CCN1 gene product or biologically active fragments thereof in cells, and specifically cancer cells. Also, some embodiments include expression cassettes under control of the CCN1/2 promoter for high cancer-specific expression of the nucleic acid-of-interest.

### SARI Gene and SARI Gene Product(s)

Suppressor of AP-1, Regulated by IFN(SARI) is a novel type I IFN-inducible early response gene that is down-regulated in a number of cancers. SARI is a gene that, based on a number of unique properties, highlights its significance in IFN-signaling. Without intending to be limited to any specific mechanism and by way of background, IFNs induce growth inhibition by multiple pathways that involve many IFN-stimulated genes (Stark G R, Kerr I M, Williams B R, Silverman R H, Schreiber R D (1998) Annu Rev Biochem 67:227-264). IFNs exert their diverse effects by stimulating the expression of a plethora of IFN-stimulated genes in target cells. Both type I (IFN-α, IFN-β, IFN-ω, and IFN-τ) and type II (IFN-γ) IFNs exert potent anti-tumor effects and are used clinically either as a monotherapy or as an adjuvant to chemotherapy or radiotherapy for a number of solid tumors and hematological malignancies that include melanoma, renal cell carcinoma, Kaposi sarcoma, malignant glioma, lymphomas, and leukemias. The anti-tumor effects of IFNs are mediated by direct inhibition of proliferation of cancer cells as well as by indirect effects such as inhibition of tumor angiogenesis and up-regulation of tumor-specific antigens and adhesion molecules.

The inventors have also found that SARI is capable of regulating promoters under the control of AP-1. Activator protein-1 (AP Activator protein-1 (AP-1), one of the first identified mammalian transcription factors, plays an essential role in regulating cell proliferation and oncogenic transformation (Angel P, Karin, M (1991) Biochim Biophys Acta 1072:129-157). AP-1 comprises a variety of dimeric basic region-leucine zipper (bZIP) proteins that belong to the Jun (c-Jun, JunB, JunD), Fos (c-Fos, FosB, Fra-1, and Fra-2), Maf, and ATF subfamilies that recognize either the 12-O-tetradecanoylphorbol-13-acetate (TPA) response element (5′-TGAG/CTCA-3′) or the cAMP response element (5′-TGACGTCA-3′) (Chinenov Y, Kerppola T K (2001) Oncogene 20:2438-2452). Among the Jun proteins, c-Jun is the most potent transcriptional activator, and the c-Fos-c-Jun heterodimer positively regulates cell proliferation and transformation (Karin supra, Maki Y, Bos T J, Davis C, Starbuck M, Vogt P K (1987) Proc Natl Acad Sci USA 84:2848-2852; and Ryseck R P, Bravo R (1991) Oncogene 6:533-542). c-Jun co-operates with Ha-ras in rat embryonic fibroblast transformation and c-jun-l-cells are refractory to the transforming activity of oncogenic Ras (Vandel L, et al. (1996) Mol Cell Biol 16:1881-1888; and Johnson R, Spiegelman B, Hanahan D, Wisdom R (1996) Mol Cell Biol 16:4504-4511). Additionally, overexpression of c-Jun itself can immortalize rodent fibroblasts in culture (Vandel, supra). Augmented AP-1 activity has been observed in more than 90% of human cancers, indicating its seminal importance in human carcinogenesis (Eferl R, Wagner E F (2003) Nat Rev Cancer 3:859-868. 24).

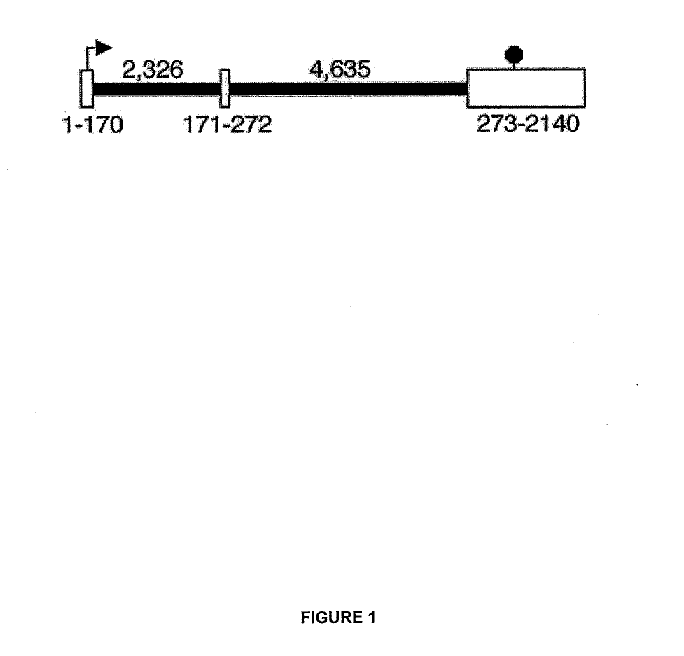

The SARI gene product is a bZIP containing protein that inhibits cell growth by interacting with c-Jun and inhibiting AP-1 activity. SARI cDNA comprises about 2,140 base pairs as depicted in SEQ ID NO. 1. The SARI gene is located in the long arm of chromosome 11, between 11q12 and 11q13. The SARI gene structures is shown in FIG. 1, and includes three exons with translation beginning in exon 1 and the stop codon located in exon 3. The SARI promoter region depicted in SEQ ID No.:4 is IFN and mezerin inducible. The minimal effective sequence of the SARI promoter ranges from about −924 to about +56. Accordingly, certain embodiments are directed to an isolated nucleic acid comprising a promoter polynucleotide substantially identical to (e.g., at least 95% identical to) the SARI promoter (SEQ. ID. No.:4) where the promoter polynucleotide initiates expression in SARI expressing cells. Furthermore, particular embodiments relate to polynucleotides comprising, or complementary to, all or part of the SARI gene, mRNA, SARI promoter region, the bZIP region, and/or SARI gene coding region. The SARI polynucleotides may be in isolated or substantially isolated form, including polynucleotides encoding SARI-related protein and biologically active fragments thereof, DNA/RNA, DNA/RNA hybrid, and related molecules, polynucleotides or oligonucleotides complementary to a SARI gene or mRNA sequence or a part thereof, and polynucleotides or oligonucleotides that hybridize to a SARI gene, mRNA, or to a SARI polynucleotide.

The SARI cDNA in SEQ ID No. 2 encodes for a putative protein of about 274-aa residues with a predicted molecular mass of about 29.4-kDa and a pI 7.2. Accordingly, certain embodiments are directed to an isolated polypeptide having an amino acid sequence that is substantially identical to (e.g., at least 65%, 70%, 75%, 80%, 85%, 90%, or 95% identical to) SEQ ID No.: 2. Further embodiments are directed to an isolated nucleic acid encoding a polypeptide substantially identical to (e.g., at least 65%, 70%, 75%, 80%, 85%, 90%, or 95% identical to) the SARI protein of SEQ ID No. 2.

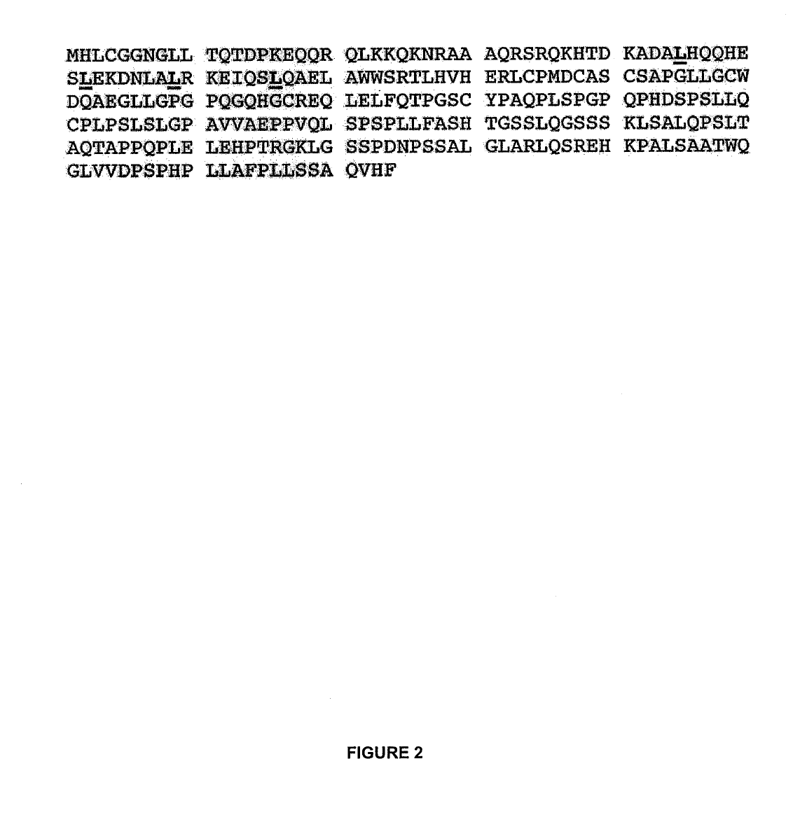

The SARI protein contains an L-X₆-L-X₆-L-X₆-L motif between 45 and 66 aa residues preceded by a highly basic region, indicating that SARI is a basic-region leucine zipper containing transcription factor (underlined sequence in FIG. 2). Accordingly, particular embodiments relate to an isolated or substantially isolated SARI gene product or biologically active fragments thereof, and specifically, the bZIP fragment, optionally preceded by a basic region. Fragments of SARI comprising the bZIP domain are useful, for example, for interfering with AP-1 interactions with other transcriptional activators and thus can be targeted (e.g., via recombinat expression) to nuclei where AP-1 interference is desirable (including but not limited to in cancer cells).

Normal cell survival is compatible with SARI expression, however, continued proliferation of cancer cells requires that SARI expression be suppressed, a phenomenon characteristic of many tumor suppressor proteins. Even when using an adenovirus-mediated delivery approach that ensures high-level gene expression in greater than about 90% of cells, SARI overexpression did not adversely affect normal cell survival, however, overexpression of SARI in cancer cells induced profound growth inhibition and apoptosis in those cells. Accordingly, certain embodiments relate to the expression (e.g., recombinant expression) or overexpression of SARI in cancer cells in vitro, in vivo, or ex vivo. Specific embodiments relate to targeted expression or overexpression of SARI for use as effective gene therapy for cancer therapy. SARI expression or overexpression as an anti-cancer agent would function by repressing or preventing the expression of specific gene products such as AEG-1, CCN1, and CCN2, for example. Accordingly, in some embodiments, SARI is recombinantly expressed or otherwise delivered or increased in cancer cells that express or over-express AEG-1, CCN1, and CCN2. SARI expression or overexpression may be implemented in a number of ways, which are detailed below.

### SARI and CCN1

CCN1 expression is regulated by SARI. CCN1 is overexpressed in many cancers such as human breast cancers, malignant gliomas, gastric adenocarcinomas, and melanoma. CCN1 is a secretory integrin-binding protein that regulates cell adhesion, migration, proliferation, survival and apoptosis (Leu S J, Chen N, Chen C C, Todorovic V, Bai T, Juric V et al. (2004) J Biol Chem 279: 44177-44187). Increased expression of CCN1 occurs in several cancer indications, i.e. human breast cancers, malignant gliomas, gastric adenocarcinomas, and melanomas (Jiang W G, Watkins G, Fodstad O, Douglas-Jones A, Mokbel K, Mansel R E (2004). Endocr Relat Cancer 11: 781-791; O'Kelly J, Chung A, Lemp N, Chumakova K, Yin D, Wang H J et al. (2008) Int J Oncol 33: 59-67). CCN1 protein is found at higher levels in breast cancer samples compared to adjacent non-tumor tissue and staining of CCN1 in tumors positively correlates with AJCC (American Joint Committee on Cancer) disease stages from I-IV.

Both in vitro and in vivo studies document that over-expression of CCN1 in breast cancer cells up-regulates MAPK signaling in an integrin αvβ3-dependent manner, thereby promoting cell survival, chemoresistance, angiogenesis and metastasis (Menendez J A, Vellon L, Mehmi I, Teng P K, Griggs D W, Lupu R (2005) Oncogene 24: 761-779; Vellon L, Menendez J A, Lupu R (2005) Oncogene 24: 3759-3773). Over-expression of CCN1 in breast cancer cell lines promotes cell growth, migration and in vivo angiogenesis (Tsai M S, Bogart D F, Castaneda J M, Li P, Lupu R (2002a) Oncogene 21: 8178-8185; Xie D, Miller C W, O'Kelly J, Nakachi K, Sakashita A, Said J W et al. (2001) J Biol Chem 276: 14187-14194). In contrast, siRNA-based gene silencing of CCN1 resulted in nearly complete inhibition of migration and invasion of breast cancer cells toward stromal fibroblasts (Nguyen N, Kuliopulos A, Graham R A, Covic L (2006). Cancer Res 66: 2658-2665). Down-regulation of CCN1 by staurosporine in prostate cancer cells is associated with neuronal differentiation and suppression of malignancy (Shimizu T, Okayama A, Inoue T, Takeda K (2005) Oncol Rep 14: 441-448.). Accordingly, particular embodiments relate to the selective inhibition of CCN1 in cancer cells to increase the therapeutic efficacy of anti-cancer agents. Inhibition of CCN1 in cancer cells may be implemented in a number of ways, further described below.

In further embodiments, an animal may be generated that expresses the CCN1 gene to generate a diagnostic cancer animal model. The animal model may be used to follow cancer development and/or to determine how the animal responds to specific therapeutic agents.

The CCN1 gene promoter contains a variant of the AP-1 consensus sequence (base −821 to −828) in which a single-base substitution of the center nucleotide has occurred (5′-TGACTCAG-3′). The AP-1 element plays a critical role in regulating CCN1 promoter activity and hence CCN1 expression is tightly regulated through the AP-1 transcription factors. The CCN1 promoter, depicted in SEQ ID No.: 5, is a very strong and highly cancer specific promoter. Accordingly, particular embodiments are directed to a promoter polynucleotide at least 95% identical to SEQ ID No.: 5 where the promoter polynucleotide initiates cancer-specific gene expression, optionally where the promoter is operably linked to another polynucleotide. Some embodiments are related to promoters, optionally operably linked to another polynucleotide, wherein the promoter comprises a fragment (e.g., at least 20, 40, 50, 75, 100, or more nucleotides) of SEQ ID NO:5, where the promoter polynucleotide initiates cancer-specific gene expression. In some embodiments, the promoter comprises a fragment of SEQ ID NO:5 that comprises the AP-1 consensus sequence. In some embodiments, the promoter can comprise a heterologous basal promoter and/or transcriptional initiation sequence linked to the AP-1 consensus sequence of the human CCN1 promoter (SEQ ID NO:5).

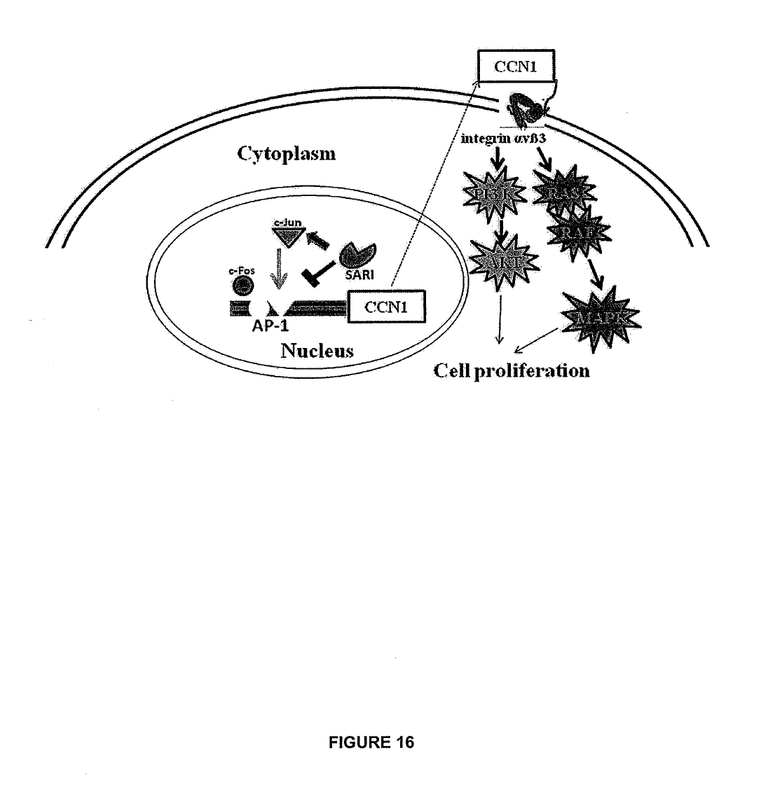

As discussed above, SARI exerts a cancer-specific anti-tumor effect by inhibiting AP-1 binding activity. The inventors have discovered that SARI selectively suppresses CCN1 transcription and hence inhibits CCN1-induced anchorage-independent growth and invasion in breast cancer, malignant glioma and metastatic melanoma cells. Additionally, it was discovered by the inventors that SARI also blocks the downstream cell proliferation signals produced by CCN1 by inhibiting the activation of MAPKinase and PI3-AKT kinases. Accordingly, SARI mediates down-regulation of CCN1 by inhibiting DNA binding of AP-1 complexes to the CCN1 promoter, thereby resulting in reduced promoter activity and consequently decreased CCN1 transcription (FIG. 16). Accordingly, certain embodiments relate to applications of SARI for therapeutic intervention in cancers that are addicted to AP-1 and its downstream targets for survival and expression of the transformed state.

In some embodiments, expression of SARI down regulates one or more of the following genes: P23/7, CD24, CD44, CD9, CD164, CD58, CD81, CD83, cyclinB1, cyclinB2, cyclinD3, cyclinE2, cyclinT2, CDC20, CDC23, CDC28, E2F, ATPsythase, RNA polymerase I, and/or metallothionein2A. Therefore, SARI expression can be directed to those cells in which reduction of expression of one or more of the above-listed genes is ameliorative. Without intending to limit the scope of the invention, it is believed that CCN1 upregulates the above-listed genes and that SARI down regulates the above-listed genes by inhibiting expression of CCN1.

### Methods of Treatment and Diagnostics

In some embodiments, methods are provided for delivering or increasing SARI expression in cancer cells (where SARI expression is reduced or not expressed and/or CCN1 is expressed or overexpressed), thereby modulating or treating the cancer or other diseases in which SARI expression is ameliorative or therapeutic. The diseases may include diseases that have an AP-1 link, such as psoriasis, inflammatory diseases, cardiovcascular diseases such as cardiac hypertrophy following hypertension, myeloproliferative diseases (Hodgkins Lymphoma, Leukemia, etc.), bone-related diseases (such as osteosclerosis, rheumatoid arthritis, and psoriatic arthritis, alcohol-induced liver inflammation, diabetic neuropathy, fibrotic disease, epidermal diseases (warts, etc.), tumor angiogenesis, and the like. In some embodiments, SARI is delivered, expressed or overexpressed in a cancer cell, or a human individual having a cancer including but not limited to breast cancer, malignant glioma and metastatic melanoma, hematopoietic malignancies (leukemia, myeloma, etc.), prostate cancer, ovarian cancer, other CNS tumors (meningioma, medulloblastoma, etc.), colon cancer, cervical cancer, esophageal cancer, thyroid cancer, lung cancer, osteosarcoma, fibrosarcoma, etc. As noted above, in some embodiments, SARI is expressed or delivered to CCN1-expressing cells. One can deliver or increase expression of SARI in cancer or other cells by any nucleic acid or protein delivery method. A number of exemplary methods are described elsewhere herein.

Other embodiments take advantage of the discovery that the human CCN1 promoter is particularly cancer-specific. Accordingly, in some embodiments, an expression cassette comprising a CCN1 promoter operably linked to a diagnostic or therapeutic protein coding sequence, or antisense or siRNA is provided. Generally, any nucleic acid-of-interest can be expressed from the CCN1 promoter. Particular embodiments generally involve targeting a nucleic acid-of-interest encoding a gene product (e.g., a nucleic acid encoding a gene product, a biologically active fragment of the gene product, RNAi, and the like) to a cell, such as a cancer cell, so that the gene product encoded by the nucleic acid-of-interest is expressed and directly or indirectly ameliorates the diseased state.

Particular embodiments are related to toxic, apoptotic, or reporter proteins can be delivered to the cancer cells, and specifically cancer cells expressing CCN1, by using expression vectors (viral or nonviral) carrying a nucleic acid under control of the cancer specific CCN1/2 promoter. Some embodiments provide expression cassettes comprising a CCN1/2 promoter operably linked to a polynucleotide encoding a protein that is cytotoxic or induces apoptosis in a target cell which include without limitation tumor suppressor genes (such as mda-7/IL-24 (see, e.g., US Patent Publication Nos. 2003/0225025 and 2006/0292157), wildtype p53, TRAIL, other suppressor genes), apoptosis-inducing genes (Bax, Bak, Femlb, tBid, etc.), and immune modulating genes (IFN-gamma, IFN-beta, mda-5, RIG-I, IL-2, IL-12, etc.). The expression vector may also carry antisense RNA gene constructs of cell cycle suppressing genes and genes regulating cell growth and survival. Moreover, the expression vector may carry siRNAs to prevent expression of CCN1 under control of the CCN1/2 promoter and thereby ameliorate the cancer which include without limitation AEG-1 shRNA and siRNA, shRNA and siRNA for oncogenic proteins such as K-Ras, Ha-Ras, N-Ras, Src, c-Jun, HPV, mda-9/syntenin, etc. Additionally, the expression vector may have a reporter gene, such as GFP, YFP or RFP under control of the CCN1/2 promoter that will allow the detection of cancer cells expressing CCN1. The reporter proteins may also include radionuclides, iodo-azomycin galactopyranoside ((131)I-IAZGP), gold or other nanoparticles, luciferase, beta-galactosidase, chloramphenical acetyltransferase, etc.

After infecting a cell, the nucleic acid-of-interest driven by a specific promoter in the vector expresses the gene product. The vector may be a viral or non-viral vector that carries the nucleic acid-of-interest. The use of the cancer specific promoter such as the CCN1/2, PEG-3, or AEG-1 promoter, will allow selective expression of the gene product in the cancer cell. In a specific embodiment, the nucleic acid-of-interest may encode the SARI gene product or a biologically active fragment thereof or an inhibitory oligonucleotide targeted to CCN1, including antisense oligonucleotides, ribozymes, short inhibitory RNA (siRNA), micro RNA (miRNA). In specific embodiments, prior to delivering an expression vector and/or therapeutic viral vector including a nucleic acid-of-interest that encodes for the SARI gene product or a biologically active fragment thereof or an expression vector and/or therapeutic viral vector including an inhibitory oligonucleotide targeted to CCN1, the cells are first screened for CCN1 and/or SARI expression.

In certain embodiments, the methods and therapeutic products of the invention may be used in combination with other conventional anti-cancer therapeutic methods directed to the treatment or prevention of proliferative disorders. For instance, these methods may encompass prevention of cancer recurrence and metastases after surgery, and as an adjuvant of other convention cancer therapies. Combinational use of conventional therapies (i.e., chemotherapy, radiation therapy, phototherapy, immunotherapy, and surgery, for example) may be enhanced or a synergistic effect may occur through the combinational or concomitant use of the therapeutic products.

Some embodiments involve isolating the nucleic acid-of interest, selecting the proper vector vehicle to deliver the nucleic acid-of-interest to the body, administering the vector having the nucleic acid-of-interest into the body, and achieving appropriate expression of the nucleic acid-of-interest. Embodiments also provide packaging the cloned genes, i.e. the genes of interest, in such a way that they can be injected directly into the bloodstream or relevant organs of patients who need them. The packaging will protect the foreign DNA from elimination by the immune system and direct it to appropriate tissues or cells. Most of the techniques used to construct vectors and the like are widely practiced in the art, and most practitioners are familiar with the standard resource materials which describe specific conditions and procedures. However, for convenience, the following sections below may serve as a guideline.

Other embodiments related to the unique properties of the cancer-specific promoters such as PEG-3 and CCN1/2, which function specifically in cancer cells with negligible or no activity in normal cells. These promoters may be used in tumor imaging and targeting (‘theranostics’) and furthermore, to be used to develop new transgenic animal models to study cancer. These promoters can be used to target tumor suppressor gene expression uniquely in cancer cells, with limited expression in normal cells, localize the expression of imaging modalities and both imaging genes (e.g., GFP) and therapeutic genes (e.g., mda-7/II-24) uniquely in primary and metastatic tumor cells, and produce transgenic mouse models of cancer permitting in vivo imaging of cancer development, progression and therapy. For example, in an embodiment, an expression cassette that includes a cancer-specific promoter operably linked to a reporter/imaging gene, which is operably linked to a therapeutic gene may be delivered and expressed in a cancer cell. In a specific embodiment, the promoter may be CCN1/2 or PEG-3, the reporter gene may be GFP, and the therapeutic gene may be mda-7/IL-24.

### Biomarkers

Specific embodiments are directed to detection of biomarkers that identify cells that are likely particularly susceptible to SARI-induced apoptosis. Exemplary biomarkers include CCN1, CCN2, and AEG-1. Accordingly, in some embodiments, an individual, or target tissue or cell is assayed for expression of one or more of these biomarkers and then, if one or more biomarkers are present, or expressed to a threshold, then the individual is identified as a good candidate for delivery of SARI. This, in some embodiments, SARI is delivered to an individual determined to have or express one or more of the biomarkers.

In another embodiment, a biomarker profile may be used for determining therapeutic efficacy or toxicity of an anti-cancer agent. If the compound has a pharmaceutical impact on the subject, organ or cell the phenotype (e.g., the pattern or profile) of the biomarkers changes towards a normal (non-cancer) profile. For example, in certain cancers, SARI expression is reduced or inhibited and CCN1 expression is increased. Therefore, one can follow the course of the amounts of these biomarkers in the subject, organ, or cell during the course of treatment. Accordingly, this method involves measuring one or more biomarkers upon prior to and after exposure to the anti-cancer agent(s). Methods for measuring the specific biomarkers are a matter of routine experimentation and are known by those of skill in the art.

### Antibodies

Certain embodiments related to anti-SARI antibodies and anti-CCN1/2 antibodies which can be used to detect endogenous and/or exogenous SARI and/or CCN1/2 proteins. Any type of antibody agonist may be used according to the methods of the invention. Generally, the antibodies used are monoclonal antibodies. Monoclonal antibodies can be generated by any method known in the art (e.g., using hybridomas, recombinant expression and/or phage display).

A number of different synthetic molecular scaffolds can also be used to display the variable light and heavy chain sequences that specifically bind the polypeptides (e.g., SARI). A publication describing use of the fibronectin type III domain (FN3) as a specific molecular scaffold on which to display peptides including CDRS is Koide, A. et al. J. Mol. Biol. 284:1141-1151 (1988). Other scaffolding alternatives include, e.g., “minibodies” (Pessi, A. et al., Nature 362:367-369 (1993)), tendamistat (McConnell, S. J. and Hoess, R. H. J. Mol. Biol. 250:460-470 (1995)), and “camelized” VH domain (Davies J. and Riechmann, L. BiolTechnology 13:475-479 (1995)). Other scaffolds that are not based on the immunoglobulin like folded structure are reviewed in Nygren, P. A. and Uhlen, M. Curr. Opin. Struct. Biol. 7:463-469 (1997). U.S. Pat. No. 6,153,380 describes additional scaffolds. The term “affinity agents” encompasses molecules comprising synthetic molecular scaffolds such as those described above to display binding domains with a binding specificity for human SARI or other mammalian SARI proteins.

In some embodiments, the antibody may be a chimeric (e.g., mouse/human) antibody made up of regions from a non-human anti-SARI antibody together with regions of human antibodies. For example, a chimeric H chain can comprise the antigen binding region of the heavy chain variable region of the non-human antibody linked to at least a portion of a human heavy chain constant region. This humanized or chimeric heavy chain may be combined with a chimeric L chain that comprises the antigen binding region of the light chain variable region of the non-human antibody linked to at least a portion of the human light chain constant region. In some embodiments, the heavy chain constant region can be an IgM or IgA antibody.

The chimeric antibodies may be monovalent, divalent, or polyvalent immunoglobulins. For example, a monovalent chimeric antibody is a dimer (HL) formed by a chimeric H chain associated through disulfide bridges with a chimeric L chain, as noted above. A divalent chimeric antibody is a tetramer (H₂ L₂) formed by two HL dimers associated through at least one disulfide bridge. A polyvalent chimeric antibody is based on an aggregation of chains.

DNA sequences of antibodies can be identified, isolated, cloned, and transferred to a prokaryotic or eukaryotic cell for expression by procedures well-known in the art. Such procedures are generally described in Sambrook et al., supra, as well as CURRENT PROTOCOLS IN MOLECULAR BIOLOGY (Ausubel et al., eds., 1989). Expression vectors and host cells suitable for expression of recombinant antibodies and humanized antibodies in particular are well known in the art. The following references are representative of methods and vectors suitable for expression of recombinant immunoglobulins which may be utilized: Weidle et al., Gene, 51: 21-29 (1987); Dorai et al., J. Immunol., 13(12):4232-4241 (1987); De Waele et al., Eur. J. Biochem., 176:287-295 (1988); Colcher et al., Cancer Res., 49:1738-1745 (1989); Wood et al., J. Immunol., 145(a):3011-3016 (1990); Bulens et al., Eur. J. Biochem., 195:235-242 (1991); Beggington et al., Biol. Technology, 10:169 (1992); King et al., Biochem. J., 281:317-323 (1992); Page et al., Biol. Technology, 2:64 (1991); King et al., Biochem. J., 290:723-729 (1993); Chaudary et al., Nature, 339:394-397 (1989); Jones et al., Nature, 321:522-525 (1986); Morrison and Oi, Adv. Immunol., 44:65-92 (1988); Benhar et al., Proc. Natl. Acad. Sci. USA, 91:12051-12055 (1994); Singer et al., J. Immunol., 150:2844-2857 (1993); Cooto et al., Hybridoma, 13(3):215-219 (1994); Queen et al., Proc. Natl. Acad. Sci. USA, 86:10029-10033 (1989); Caron et al., Cancer Res., 32:6761-6767 (1992); Cotoma et al., J. Immunol. Meth., 152:89-109 (1992). Moreover, vectors suitable for expression of recombinant antibodies are commercially available.

Host cells capable of expressing functional immunoglobulins include, e.g., mammalian cells such as Chinese Hamster Ovary (CHO) cells; COS cells; myeloma cells, such as NSO and SP2/O cells; bacteria such as Escherichia coli; yeast cells such as Saccharomyces cerevisiae; and other host cells.

In some embodiments, the antibodies are single chain antibodies. Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., Proc. Natl. Acad. Sci. USA 90:7995-7999 (1993); and Skerra et al., Science 240:1038-1040 (1988).

In some embodiments, human antibodies may be used according to particular embodiments. Human antibodies can be made by a variety of methods known in the art including by using phage display methods using antibody libraries derived from human immunoglobulin sequences. See, e.g., Lonberg and Huszar, Int. Rev. Immunol. 13:65-93 (1995), U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

In some embodiments, the antibodies may be generated using phage display. For example, functional antibody domains are displayed on the surface of phage particles that carry the polynucleotide sequences encoding them. Such phage can be utilized to display antigen-binding domains expressed from a repertoire or combinatorial antibody library (e.g., human or murine). Phage expressing an antigen binding domain that binds SARI can be selected or identified with SARI, e.g., using labeled SARI or a portion thereof. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies may include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

### General Molecular Biology Techniques

The following molecular biology techniques are well known by those of ordinary skill in the art and are available in text commonly used by those of ordinary skill in the art such as Molecular Cloning: A Laboratory Manual, 3^{rd} Ed., CSHL Press 2001; and Current Protocols in Molecular Biology, John Wiley & Sons, Inc., 2007, the entire disclosures of which are herein expressly incorporated by reference in their entirety.

Construction of Nucleic Acids

The isolated nucleic acids of the invention can be made using (a) recombinant methods, (b) synthetic techniques, (c) purification techniques, or combinations thereof, as well-known in the art.

The nucleic acids may include sequences in addition to a polynucleotide. For example, a multi-cloning site comprising one or more endonuclease restriction sites can be inserted into the nucleic acid to aid in isolation of the polynucleotide. Also, translatable sequences may be inserted to aid in the isolation of the translated polynucleotide. For example, a hexa-histidine marker sequence provides a convenient means to purify the proteins. The nucleic acids—excluding the coding sequence—may be optionally a vector, adapter, or linker for cloning and/or expression of a polynucleotide.

Additional sequences may be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art.

Recombinant Methods for Constructing Nucleic Acids

The isolated nucleic acid compositions, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides may be used to identify the desired sequence in a cDNA or genomic DNA library. The isolation of RNA, and construction of cDNA and genomic libraries, is well known to those of ordinary skill in the art.

Nucleic Acid Screening and Isolation Methods

A cDNA or genomic library can be screened using a probe based upon the sequence of a polynucleotide, such as those disclosed herein. Probes can be used to hybridize with genomic DNA or cDNA sequences to isolate homologous genes in the same or different organisms. Those of ordinary skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of about 0% to about 50%. The degree of complementarity (sequence identity) required for detectable binding will vary in accordance with the stringency of the hybridization medium and/or wash medium. The degree of complementarity may be about 100%, or about 70% to about 100%, or any range or value therein. However, it should be understood that minor sequence variations in the probes and primers may be compensated for by reducing the stringency of the hybridization and/or wash medium.

Methods of amplification of RNA or DNA are well known in the art and can be used according to the invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. No. 4,795,699 and U.S. Pat. No. 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the disclosures of all references in this paragraph are incorporated by reference herein in their entirety.

For example, polymerase chain reaction (PCR) technology may be used to amplify the sequences of polynucleotides and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

Synthetic Methods for Constructing Nucleic Acids

The isolated nucleic acids may also be prepared by direct chemical synthesis by known methods. Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

Embodiments further provide recombinant expression cassettes having a nucleic acid-of-interest. A nucleic acid-of-interest may include, for example, a nucleic acid sequence that expresses cancer toxic gene (e.g., mda-7 as disclosed in U.S. Pat. Nos. 7,507,792, 7,291,605, 6,982,148, and 6,720,408), reporter genes (e.g., GFP), antisense RNA, RNAi to inhibit expression of a desired gene product such as CCN1 or SARI. In specific embodiments, the nucleic acid-of-interest may encode SARI or biologically active fragments thereof, such as the SARI bZIP region. Certain embodiments are also directed to producing SARI knock-out cells or animals. This may be accomplished by expressing an siRNA complementary to all or a portion of a mRNA having SEQ ID No.: 3 and encoding a SARI protein, to inhibit the expression of SARI.

The nucleic acid-of-interest may be used to construct a recombinant expression cassette that can be introduced into at least one desired host cell. A recombinant expression cassette will typically comprise a polynucleotide operably linked to transcriptional initiation regulatory sequences that will direct the transcription of the polynucleotide in the intended host cell. Both heterologous and non-heterologous (i.e., endogenous) promoters can be employed to direct expression of the nucleic acids.

In some embodiments, isolated nucleic acids that serve as promoter, enhancer, or other elements may be introduced in the appropriate position (upstream, downstream or in intron) of a non-heterologous form of a polynucleotide so as to up or down regulate expression of a polynucleotide. For example, endogenous promoters can be altered in vivo or in vitro by mutation, deletion and/or substitution. In specific embodiments, the promoter sequences comprises the SARI promoter encoded by SEQ ID NO.: 4, the CCN1 promoter encoded by SEQ ID NO.: 5, or the PEG-3 promoter encoded by SEQ ID NO.: 6, or active fragments or variants (e.g., substantially identical) thereof. The promoter sequences may be full length or active fragments thereof that retain the expression pattern. In some cases, the fragments can be at least 50, 100, 150, 200, 250, 300, 350, 400, 450, 500, 550, 600, 650, 700, 750, 800, and 850, base pairs.

In specific embodiments, the expression cassette can have a nucleic acid-of-interest under control of the CCN1/2 promoter. The nucleic acid-of-interest under control of the CCN1/2 promoter can be a reporter protein (including but not limited to green fluorescent protein or other fluorescent proteins) to be used as a cancer diagnostic, an anti-cancer protein, such as mda-7 or SARI, or an antisense RNA or RNAi (including but not limited to antisense RNAs or RNA is directed to blocking expression of CCN1), for example as described elsewhere herein.

In some embodiments, the SARI promoter is operably linked to a detectable gene product and expressed in cells (e.g., mammalian cells). Libraries of molecules can then be ioncubated with the cells to identify molecules that activate SARI promoter expression. Such molecules can be further developed as agents that induce SARI expression in cancer cells.

Vectors

Certain embodiments also relate to vectors that include isolated nucleic acid molecules, host cells that are genetically engineered with the recombinant vectors, and the production the SARI gene product or biologically active fragments thereof, and inhibitory oligonucleotides (e.g., antisense RNA or RNAi) to prevent the expression of the CCN1 gene product, by recombinant techniques, as is well known in the art.

The polynucleotides may optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter, which may be the SARI, CCN1, CCN2, PEG-3, or AEG-1 promoters or the regions of the promoters that are capable of driving gene expression. The expression constructs will further contain sites for transcription initiation, termination and, in the transcribed region, a ribosome binding site for translation. The coding portion of the mature transcripts expressed by the constructs may include a translation initiating at the beginning and a termination codon (e.g., UAA, UGA or UAG) appropriately positioned at the end of the mRNA to be translated.

Expression vectors may include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, colormetric markers such as GFP and beta-galactosidase, ampicillin, neomycin (G418), mycophenolic acid, or glutamine synthetase (U.S. Pat. Nos. 5,122,464; 5,770,359; 5,827,739) resistance for eukaryotic cell culture, and tetracycline or ampicillin resistance genes for culturing in E. coli and other bacteria or prokaryotics. Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods.

Those of ordinary skill in the art are knowledgeable in the numerous expression systems available for expression of a nucleic acid encoding a protein, and thus there is no need to describe them herein.

### Gene Therapy/Expression Vectors

Conventional viral and non-viral based gene transfer methods can be used to introduce expression cassettes (to deliver proteins or inhibitory nucleic acids such as antisense or siRNAs) in mammalian cells or target tissues. Such methods can be used to deliver expression cassettes to cells in vitro. In some embodiments, the expression cassettes are administered for in vivo or ex vivo gene therapy uses. Non-viral vector delivery systems include DNA plasmids, naked nucleic acid, and nucleic acid complexed with a delivery vehicle such as a liposome. Additional delivery systems may include microbubbles systems as disclosed in U.S. Pat. No. 7,115,583 or nanoparticle delivery systems as disclosed in U.S. Pat. No. 7,588,778. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

Methods of non-viral delivery of nucleic acids encoding engineered polypeptides include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of expression cassettes of take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In some applications where transient expression from the expression cassettes is desired, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a possible alternative gene delivery systems based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiply types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998). In specific embodiments, the adenovirus vector is a bipartite viral vector comprising the CCN1 promoter operably linked to, and controlling expression of, a gene necessary for viral replication, wherein the viral vector also encodes a second gene product that is toxic to, or induces apoptosis in a diseased cell. The second gene product can be controlled by a disease specific or other promoter. Genes necessary for viral replication are generally known and depend on the viral vector used. An examples of adenoviral genes necessary for replication ateh E1A and E1B gene products. With this bipartite construct, the CCN1 promoter drives the replication in cancer cells resulting in the production of a second therapeutic gene product, e.g., including without limitation a tumor suppressor gene (such as mda-7/IL-24, wildtype p53, TRAIL, other suppressor genes), an apoptosis-inducing gene (Bax, Bak, Fem1b, tBid, etc.), or an immune modulating gene (IFN-gamma, IFN-beta, mda-5, RIG-I, IL-2, IL-12, etc.) or antisense or siRNA molecules as discussed herein. Bipartite adenoviruses are known in the art and are generally disclosed in, e.g., U.S. Pat. No. 6,638,762 as well as, e.g., Sarkar, Su, and Fisher, Cell Cycle 5(14): 1531-1536 (2006).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In some embodiments, cells are isolated from the subject organism, transfected with a nucleic acid comprising an expression cassette, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. In a non-limiting example, methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells can be isolated for transduction and differentiation using known methods. For example, stem cells can be isolated from bone marrow cells by panning the bone marrow cells with antibodies which bind unwanted cells, such as CD4+ and CD8+ (T cells), CD45+ (panB cells), GR-1 (granulocytes), and Tad (differentiated antigen presenting cells) (see Inaba et al., J. Exp. Med. 176:1693-1702 (1992)). Alternatively, induced pluripotent stem cells can be used.

Vectors (e.g., retroviruses, adenoviruses, liposomes, etc.) containing the expression cassettes can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions, as described below (see, e.g., Remington's Pharmaceutical Sciences, 17th ed., 1989).

### Inhibitory Oligonucleotides

In some embodiments, the one or more agents are inhibitory oligonucleotides, including antisense oligonucleotides, ribozymes, short inhibitory RNA (siRNA), micro RNA (miRNA). Libraries of randomized oligonucleotides are commercially available from, for example, Integrated DNA Technologies (IDT), Coralville, Iowa; Ambion, Austin, Tex. and Qiagen, Valencia, Calif. In some embodiments, expression cassettes encode an inhibitory oligonucleotide that inhibits SARI expression. Alternatively, the embodiments also provide for expression cassettes comprising, e.g., SARI or CCN1 promoter sequences operably linked to any inhibitory oligonucleotide for inhibiting a target gene product's expression.

Antisense Oligonucleotides

An “antisense” oligonucleotide corresponds to an RNA sequence as well as a DNA sequence coding therefor, which is sufficiently complementary to a particular mRNA molecule, for which the antisense RNA is specific, to cause molecular hybridization between the antisense RNA and the mRNA such that translation of the mRNA is inhibited. Such hybridization can occur under in vitro and in vivo conditions. The antisense molecule must have sufficient complementarity to the target gene so that the antisense RNA can hybridize to the target gene (or mRNA) and inhibit target gene expression regardless of whether the action is at the level of splicing, transcription, or translation. In some embodiments, the complementary antisense sequence is about 15-30 nucleotides in length, for example, 15, 16, 17, 18, 19, 20, 21, 22, 23, 24, 25, 26, 27, 28, 29 or 30 nucleotides, or longer or shorter, as desired. The antisense components of the may be hybridizable to any of several portions of the target cDNA, including the coding sequence, 3′ or 5′ untranslated regions, or other intronic sequences, or to target mRNA.

Antisense oligonucleotides can include sequences hybridizable to any of several portions of the target DNA, including the coding sequence, 3′ or 5′ untranslated regions, or other intronic sequences, or to target mRNA.

Small Inhibitory RNA Oligonucleotides

siRNA technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. siRNA can be effected by introduction or expression of relatively short homologous dsRNAs. For screening purposes, the double stranded oligonucleotides used to effect inhibition of expression, at either the transcriptional or translational level, can be of any convenient length. siRNA molecules are typically from about 15 to about 30 nucleic acids in length, for example, about 19-25 nucleic acids in length, for example, about 15, 16, 17, 18, 19, 20, 21, 22, 23, 24, 25, 26, 27, 28, 29, 30 nucleic acids in length. Optionally the dsRNA oligonucleotides can include 3′ overhang ends. Exemplary 2-nucleotide 3′ overhangs can be composed of ribonucleotide residues of any type and can be composed of 2′-deoxythymidine resides, which lowers the cost of RNA synthesis and can enhance nuclease resistance of siRNAs in the cell culture medium and within transfected cells (see, Elbashi et al., 2001, Nature 411:494-8).

Longer dsRNAs of 50, 75, 100 or even 500 base pairs or more can also be utilized in certain embodiments. Exemplary concentrations of dsRNAs for effecting inhibition are about 0.05 nM, 0.1 nM, 0.5 nM, 1.0 nM, 1.5 nM, 25 nM or 100 nM, although other concentrations can be utilized depending upon the nature of the cells treated, the gene target and other factors readily discernable to the skilled artisan.

Exemplary dsRNAs can be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see, e.g., Elbashir, et al., 2001, Genes Dev. 15:188-200). Alternatively the dsRNAs can be transcribed from a mammalian expression vector. A single RNA target, placed in both possible orientations downstream of an appropriate promoter for use in mammalian cells, will transcribe both strands of the target to create a dsRNA oligonucleotide of the desired target sequence. Any of the above RNA species will be designed to include a portion of nucleic acid sequence represented in a target nucleic acid.

The specific sequence utilized in design of the siRNA oligonucleotides can be any contiguous sequence of nucleotides contained within the expressed gene message of the target. Programs and algorithms, known in the art, may be used to select appropriate target sequences. See, the Ambion website at ambion.com. In addition, optimal sequences can be selected utilizing programs designed to predict the secondary structure of a specified single stranded nucleic acid sequence and allowing selection of those sequences likely to occur in exposed single stranded regions of a folded mRNA. Methods and compositions for designing appropriate siRNA oligonucleotides may be found, for example, in U.S. Pat. No. 6,251,588, the contents of which are incorporated herein by reference.

Particular embodiments provide a precursor RNAi molecule capable of mediating the expression of a SARI or CCN1-specific mRNA to which it corresponds having a first oligonucleotide strand that has a length in a range of about 15 nucleotides to about 30 nucleotides; and a second oligonucleotide strand that has a length of about 15 nucleotides to about 30 nucleotides and has a nucleotide sequence that is sufficiently complementary to a sequence of an RNA of the target gene to direct target-specific RNAi. The second oligonucleotide strand anneals to the first oligonucleotide strand under biological conditions. The RNAi molecule mediates RNA interference of a adenovirus-specific mRNA to which it corresponds. In one embodiment, the precursor RNAi molecule targets the gene that encodes SARI and/or CCN1 gene products.

Certain embodiments also provide an expression cassette containing a nucleic acid encoding at least one strand of the precursor RNAi molecule described above. The expression cassette may further contain a promoter, such as inducible promote, a viral promoter, or a constitutive promoter. In certain embodiments the promoter may be a cytomegalovirus (CMV), rous sarcoma virus (RSV), pol II, or pol III promoter. In certain embodiments, the expression cassette may further contain a polyadenylation signal, such as a synthetic minimal polyadenylation signal. In certain embodiments, the expression cassette further contains a marker gene. Specific embodiments provide a cell containing the expression cassette. The cell may be a mammalian cell. Other embodiments further provide a non-human mammal containing the expression cassette.

Some embodiments also provide a vector containing the expression cassette described above. The vector, in some embodiments, may contain two expression cassettes: a first expression cassette having a nucleic acid encoding the first strand of the RNA duplex, and a second expression cassette having a nucleic acid encoding the second strand of the RNA duplex. Particular embodiments provide a vector containing an expression cassette having (1) a nucleic acid sequence encoding a first portion of RNA, (2) a second portion of RNA located immediately 3′ of the first portion of RNA, and (3) a third portion of RNA located immediately 3′ of the second portion of RNA, where the first and third portions of RNA are each less than about 30 nucleotides in length and each more than about 15 nucleotides in length. The sequence of the third portion of RNA may be the complement of the sequence of the first portion of RNA to form an RNA duplex. The RNA duplex may mediate RNA interference of a adenovirus type 5 mRNA to which it corresponds. The vectors described above may further contain a polyadenylation signal and/or a marker gene. The vector may be an adenoviral, lentiviral, adeno-associated viral (AAV), poliovirus, HSV, or murine Moloney-based viral vector. Alternatively, the vector may be a plasmid vector. Some embodiments provide a composition of a polymer or excipient and the vector.

Ribozymes

Ribozymes are enzymatic RNA molecules capable of catalyzing specific cleavage of RNA. The composition of ribozyme molecules can include one or more sequences complementary to a target mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Inhibitory oligonucleotides can be delivered to a cell by direct transfection or transfection and expression via an expression vector. Appropriate expression vectors include mammalian expression vectors and viral vectors, into which has been cloned an inhibitory oligonucleotide with the appropriate regulatory sequences including a promoter to result in expression of the antisense RNA in a host cell. Suitable promoters can be constitutive or development-specific promoters. Transfection delivery can be achieved by liposomal transfection reagents, known in the art (e.g., Xtreme transfection reagent, Roche, Alameda, Calif.; Lipofectamine formulations, Invitrogen, Carlsbad, Calif.). Delivery mediated by cationic liposomes, by retroviral vectors and direct delivery are efficient. Another possible delivery mode is targeting using antibody to cell surface markers for the target cells.

Without further elaboration, it is believed that one skilled in the art using the preceding description can utilize the invention to the fullest extent. The following examples are illustrative only, and not limiting of the disclosure in any way whatsoever.

### EXAMPLES

### Specific Example 1

### SARI is a Type I MN-Inducible Early Response Gene

Gene expression analysis identified SARI as a type I IFN (IFN-α/β-inducible early response gene. In HO-1 human melanoma cells and SV40 T/tAg-immortalized primary human melanocytes (FM516-SV), SARI mRNA expression could be detected as early as 2 h after IFN-β treatment (100 U/ml) with maximum induction at 12 h post-treatment with a gradual decrease thereafter and a return by 48 h to the basal level of expression. The induction was detected with as little as 1 U/ml of IFN-β. Interestingly, in FM516-SV cells, but not in HO-1 melanoma cells, SARI expression could be detected under de novo conditions. A similar expression and induction profile could be detected in a series of normal and cancer cells from diverse tissue origins. More importantly, under basal conditions, SARI mRNA expression could be detected in multiple normal cells, including primary human fetal astrocytes (PHFAs), immortal normal mammary epithelial cells (HBL100), immortal prostate epithelial cells (P69), and immortal pancreatic mesothelial cells (LT2), but not in their malignant counterparts. Quantitative RT-PCR confirmed that SARI mRNA expression was significantly higher in PHFA versus T98G malignant glioma cells, P69 versus DU145 prostate cancer cells, as well as FM516-SV versus HO-1 melanoma cells. Treatment with cycloheximide, a protein translation inhibitor, did not by itself induce SARI mRNA expression and moderately increased IFN-β-mediated induction, suggesting that IFN-β predominantly regulates SARI expression on a transcriptional level. The half-life of the IFN-β-inducible transcript was about 2 h as revealed by actinomycin D treatment, which prevents new RNA transcription. Multiple tissue Northern blots revealed SARI expression in diverse tissues, with highest expression in pancreas and spleen and moderate expression in colon, heart, kidney, liver, lung, and prostate. Low-level expression could be detected in placenta, stomach, small intestine, and salivary gland. Brain, muscle, and testis were conspicuous by the absence of SARI expression.

### Specific Example 2

### SARI Inhibits Growth of Cancer Cells In Vitro

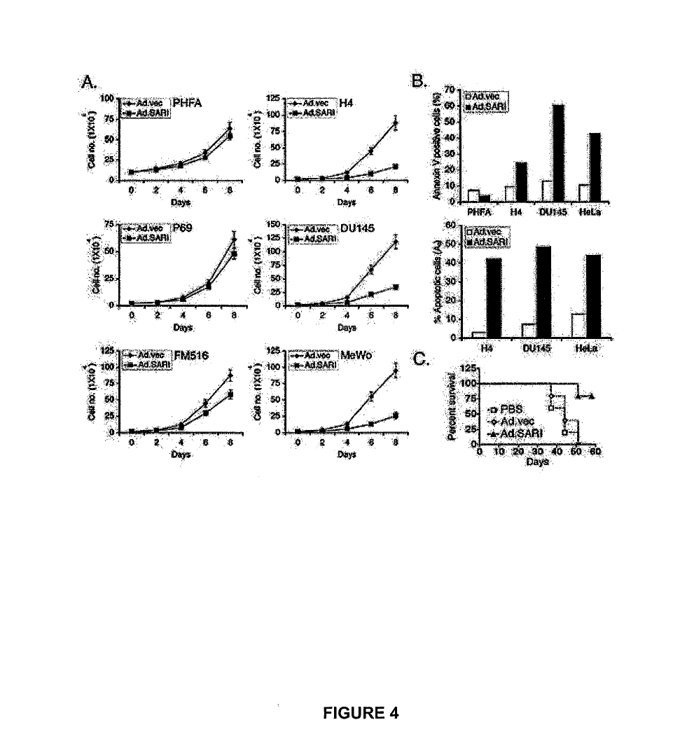

SARI inhibits growth of cancer cells but not the growth of normal cells. A replication-incompetent adenovirus expressing SARI (Ad.SARI) was constructed. PHFA, H4 (malignant glioma), P69, DU-145, FM516-SV, and MeWo (i.e., melanoma) cells were infected with Ad.SARI (100 pfu/cell) and cell growth was monitored by viable cell counting. Empty adenovirus (Ad.vec) served as a control. Ad.SARI profoundly inhibited cancer cell growth (H4, DU145, and MeWo) with little to no effect on normal cells (PHFA, P69, and FM516-SV; FIG. 4A). It should be noted that the expression level of SARI upon Ad.SARI infection was equivalent in all cell lines, as confirmed by Western blot analysis using anti-SAR1 antibody. To determine if this inhibition of growth was a result of induction of apoptosis, flow cytometry studies were performed to follow Annexin V staining or propidium iodide staining to identify subG₁ (A₀) cell populations. It was deteremined that Ad.SARI, not Ad.vec, resulted in a significant induction of apoptosis in cancer cells (H4, DU145, and HeLa), but not in PHFA normal cells (FIG. 4B).

### Specific Example 3

### SARI Inhibits Cancer Growth In Vivo

SARI inhibits cancer growth in vivo. Subcutaneous xenografts from DU-145 prostate cancer cells were established in the left flank of athymic nude mice. When the tumors reached about 100 mm³ in size, requiring about 7 days, intratumoral injections of PBS solution, Ad.vec, or Ad.SARI (10⁸ pfu/injection) were administered three times during the first week and then twice weekly for two more weeks for a total of seven injection. Tumor growth was significantly inhibited by Ad.SARI treatment compared with PBS or Ad.vec treatment. Monitoring animal survival demonstrated that Ad.SARI significantly prolonged survival compared with PBS-treated or Ad.vec-infected animals, confirming the tumor-suppressor properties of SARI in vivo (FIG. 4C).

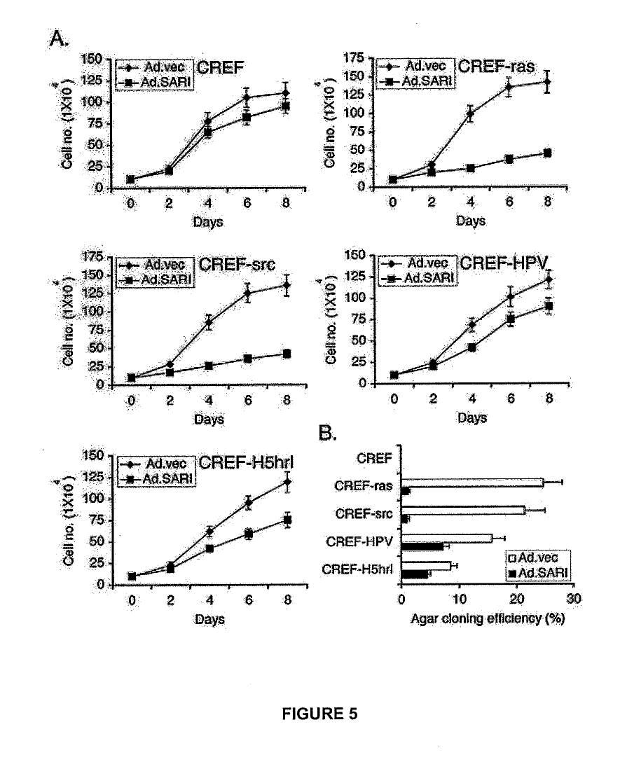

To interrogate the molecular pathway(s) involved in Ad.SARI-mediated growth inhibition we employed normal immortal rat embryonic fibroblasts (CREF) and a clone of CREF transformed by H-ras, v-src, HPV-18 or a specific temperature (cold) sensitive mutant of type 5 adenovirus H5hrl (CREF-ras, CREF-src, CREF-HPV and CREF-H5hrl, respectively). The parental CREF do not grow in soft agar and are non-tumorigenic while its transformed clones all grow in soft agar and are tumorigenic. Ad.SARI had no discernible effect on the growth of CREF. Among the tumorigenic clones a preferential inhibition of growth was observed in CREF-ras and CREF-src clones with a significantly reduced effect on CREF-HPV and CREF-H5hrl clones (FIG. 5A). These findings were bolstered by clonogenic assays as well as by soft agar assays (FIG. 5B and data not shown). These results indicate that signaling pathways, activated by ras and src, but not by nuclear-acting oncogenes such as HPV-18 and H5hrl, make CREF susceptible to Ad.SARI-mediated growth inhibition.

### Specific Example 4

### SARI Mediates IFN-β-Induced Growth Inhibition

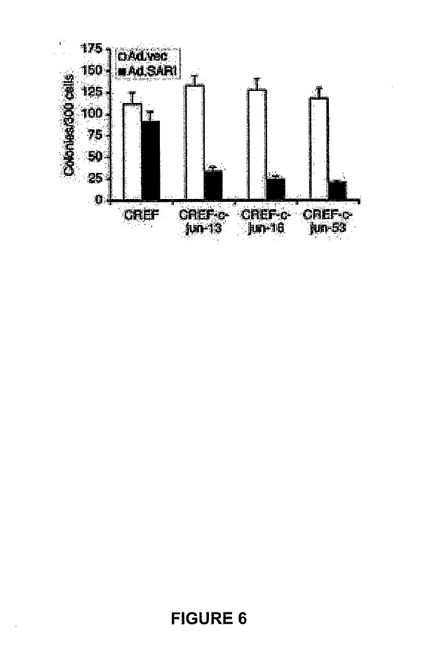

Bioinformatics analysis of SARI predicted a putative basic leucine zipper motif that might contain a c-JUN-dimerization motif. As c-JUN, a basic leucine zipper containing protein, is a component of the AP-1 transcription factor, and both ras- and src-mediated signaling result in AP-1 activation. This example shows that SARI interacts with c-JUN, thereby interfering with AP-1 function and inhibiting growth of cells dependent on AP-1 for proliferation and survival (e.g., CREF-ras and CREF-src). CREF were resistant to growth suppression by Ad.SARI, whereas CREF stably overexpressing c-jun became susceptible to Ad.SARI-mediated growth inhibition FIG. 6).

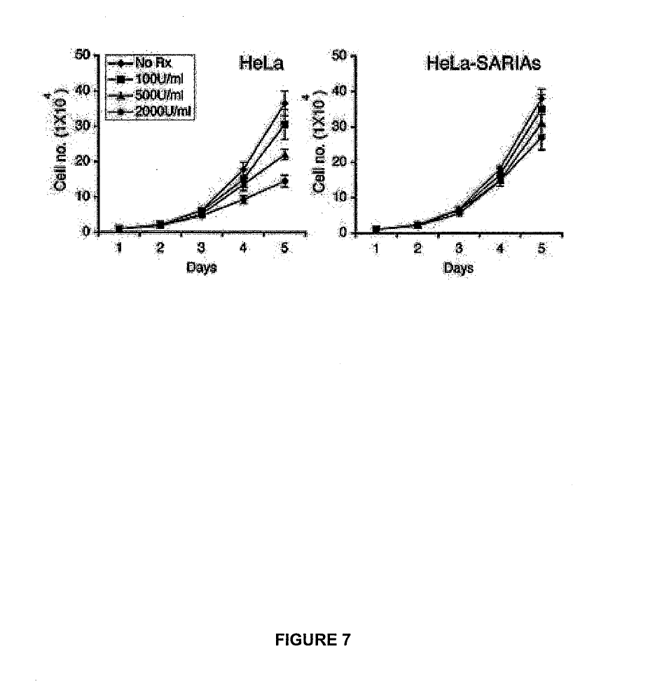

Stable HeLa cells expressing antisense SARI (HeLa-SARIAs) were established, expression and TN-β induction of SARI protein in these clones was analyzed. Cytoplasmic and nuclear extracts were prepared from parental HeLa cells, a control HeLa clone not expressing SARIAs, and HeLa-SARIAs clones untreated or treated with IFN-β. SARI protein was detected exclusively in the nucleus and induction of SARI protein by IFN-β was observed in HeLa cells and in the control clone, but not in HeLa-SARIAs clones, confirming the authenticity of these clones. Growth of HeLa and HeLa-SARIAs cells were evaluated upon IFN-β treatment. HeLa-SARIAs cells were more resistant to growth inhibition by IFN-β compared with parental HeLa cells, indicating that SARI plays an important role in mediating IFN-β action (FIG. 7).

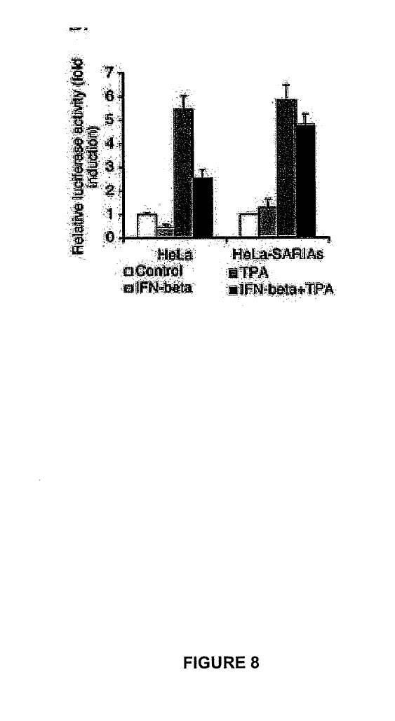

Parental HeLa and HeLa-SARIAs cells were transfected with an AP-1 reporter plasmid, in which the luciferase gene is regulated by consensus AP-1-binding sites (AP-1-luc), and then treated with TPA, IFN-β, or both. HeLa and HeLa-SARIAs cells responded equally to TPA for AP-1-luc induction (FIG. 8). However, IFN-β significantly reduced both basal and TPA-induced activation of AP-1-luc only in HeLa cells. HeLa-SARIAs cells displayed significant resistance to the inhibitory effect of IFN-β (FIG. 8). TPA efficiently activated AP-1-luc activity. However, SARI, upon induction by IFN-β, interfered with TPA-mediated AP-1-luc activation that could be rescued by antisense inhibition of SARI induction. These findings were further confirmed by electrophoretic mobility shift assay (EMSA) using nuclear extracts from TPA- or TPA/IFN-β-treated HeLa and HeLa-SARIAs cells and a radiolabeled consensus AP-1 probe. In HeLa cells, TPA-induced augmentation of AP-1-binding activity was markedly reduced by IFN-β treatment. This inhibition by IFN-β3 was profoundly abrogated in HeLa-SARIAs cells. These findings indicate that SARI plays a key role in mediating IFN-β-mediated down-regulation of AP-1 activity.

The inhibition of AP-1 activity by SARI was reflected in the expression level of an AP-1 downstream gene, IL-8. TPA treatment resulted in IL-8 mRNA induction in control and Ad.vec-infected HeLa cells. However, upon infection with Ad.SARI, TPA-mediated induction of IL-8 mRNA was markedly reduced. The steady-state expressions of cell cycle regulatory proteins such as cyclin D1 and cyclin E, which are downstream of AP-1, were also significantly down-regulated upon Ad.SARI infection.

### Specific Example 5

### SARI Interacts with c-JUN

Potential interactions between c-JUN and SARI were evaluated using multiple approaches, (i) a mammalian two-hybrid assay in which plasmid pCMV-AD was used for cloning the c-jun cDNA to be expressed as a fusion with the activation domain of VP16 (pAD-c-jun) and (ii) plasmid pCMV-BD was used to clone the SARI cDNA to be expressed as a fusion with the DNA-binding domain of GAL4 (pBD-SARI).

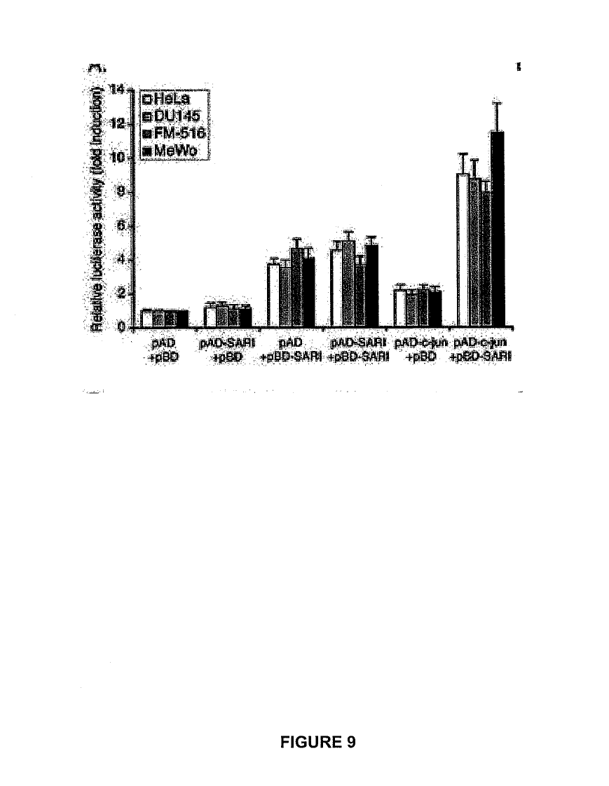

These constructs were transfected along with a reporter vector that contains five GAL4 binding sites (GAL4UAS) upstream of a minimal TATA box promoter driving the expression of a luciferase reporter gene in HeLa, DU145, FM516-SV, and MeWo cells. Interaction between SARI and c-JUN resulted in the association of the GAL4 DNA binding domain with the VP16 transcription activation domain and a significant induction of luciferase activity was observed only when pAD-c-jun and pBD-SARI were transfected together (FIG. 9). HeLa cells were infected with Ad.SARI and the cells were processed for dual immunofluorescence analysis using anti-HA antibody to detect HA-tagged SARI and anti-c-Jun antibody to detect c-Jun. Both SARI (green) and c-JUN (red) were detected in the nucleus and the merged images showed overlapping distribution of green, red, and blue (DAPI detecting nucleus), producing a white color. Co-immunoprecipitation analysis using SARI-overexpressing HeLa cell lysates and anti-HA and anti-c-JUN antibodies confirmed their interaction. Anti-HA and anti-c-JUN antibodies could effectively pull down c-JUN and SARI, respectively. It should be noted that similar assays did not find any interaction between SARI and either c-Fos or ATF-2, indicating that the interaction of SARI with c-Jun is specific.

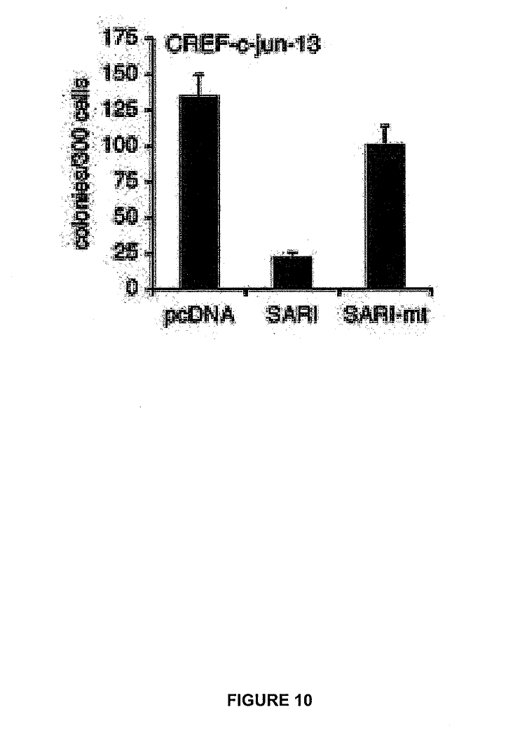

Mutation of leucine in the bZIP domain of SARI into proline completely inhibited interaction of c-JUN and SARI, indicating that the leucine zipper motif mediates the interaction between c-JUN and SARI. WT SARI could significantly inhibit colony formation of CREF-c-Jun-13 cells, the mutant SARI almost completely lost this activity, indicating that growth inhibition by SARI is mediated by its interaction with c-JUN (FIG. 10).

### Specific Example 6

### SARI Inhibits CCN1-Induced Anchorage-Independent Growth and Invasion in Cancer Cells

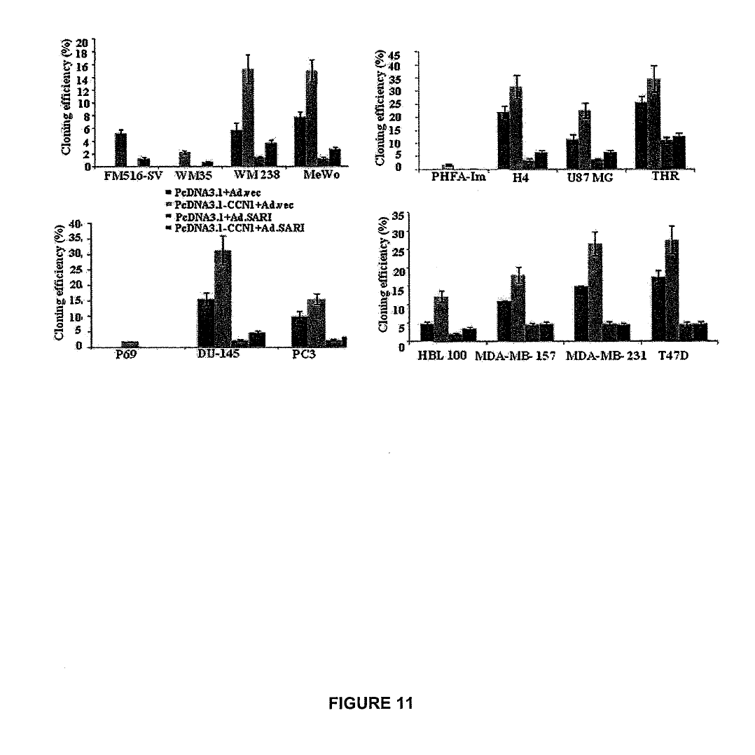

A direct relationship between CCN1 expression and increased cell growth, proliferation and invasion has been observed. This example demonstrates that transient transfection with a CCN1 expression construct enhanced anchorage-independent growth in both immortal normal and cancer cell lines from different lineages, including normal immortal melanocyte/melanoma, normal immortal primary human fetal astrocyte/malignant glioma, normal immortal prostate epithelial/prostate carcinoma and normal immortal human breast epithelial/breast carcinoma. In contrast, infection with an adenovirus that expresses SARI (Ad.SARI) at a low dose (10 pfu/cell) inhibited agar growth in all tumor cell contexts. Additionally, the ability of CCN1 to stimulate anchorage-independence was also inhibited when cells were simultaneously infected with Ad.SARI (10 pfu/cell) (FIG. 11).

The tumorigenic properties of cancer cells are facilitated by their ability to migrate and invade surrounding tissue. Using a Matrigel invasion assay, overexpression of CCN1 enhanced invasion in both weakly aggressive (MDA-MB-453, breast carcinoma; WM35, radial growth phase primary melanoma) and highly aggressive (MDA-MB-231, breast carcinoma; WM238, metastatic melanoma) cancer cells. As observed with anchorage-independent growth, infection with SARI reduced CCN1-induced invasive ability of both weakly and highly aggressive breast cancer and melanoma cells.

### Specific Example 7

### Forced Expression of SARI Down-Regulates CCN1 Expression by Inhibiting Transcription

This example demonstrates that SARI inhibits CCN1-induced expression of transformation-associated properties. First, expression of CCN1 in the presence of SARI at a transcriptional and translational level following treatment with 12-0-tetradecanoyl-phorbol-13-acetate (TPA) was determined. Infection with low dose Ad.SARI (20 pfu/cell) inhibited baseline and TPA-induced CCN1 expression both at an mRNA and protein level in human melanomas, malignant gliomas and breast cancer cells. Using nuclear run-on assays, that measure the rate of new primary transcript formation, over expression of SARI inhibited the transcription of CCN1 message (mRNA) in a time-dependent manner, evident as early as 12 h post-infection with Ad.SARI. Following infection with Ad.SARI (20 pfu/cell), significant suppression of CCN1 protein expression was observed in a temporal manner in all of the cancer cell lines, with complete extinction of CCN1 protein expression in specific cancer/transformed cell lines by 24 to 48 h. Similarly, a time-dependent inhibition in CCN1 mRNA expression was observed following infection with Ad.SARI (data not shown).

### Specific Example 8

### SARI Attenuates CCN1 Transformation-Associated Properties by Inhibiting CCN1 Expression and its Downstream Genes in Breast Cancer Cells

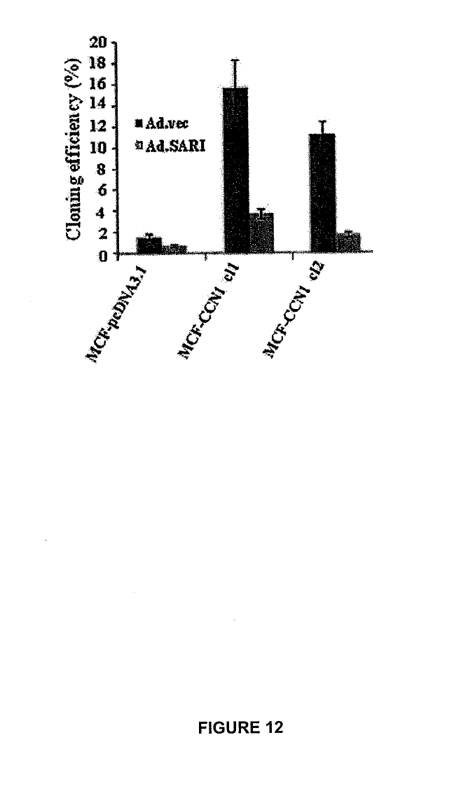

This example investigated investigate the role of SARI in modulating CCN1 expression by generating stable MCF-7 breast carcinoma clones constitutively expressing elevated levels of CCN1 (FIG. 12). MCF-7 cells were used for these studies because they express low levels of CCN1 endogenously as compared to other breast carcinoma cells (FIG. 12 for pcDNA3.1, vector DNA transformed MCF-7 clone; and data not shown). Stable MCF-7 CCN1 clones (MCF-CCN1 cl1 and MCF-CCN1 cl2) displayed elevated CCN1 expression in comparison with a parental pcDNA3.1 transfected control MCF-7 clone (MCF-pcDNA3.1). Infection with Ad.SARI (20 pfu/cell) decreased CCN1 protein levels in control and the CCN1 over-expressing MCF-7 clones). Stable elevated expression of CCN1 in MCF-7 cells resulted in enhanced phosphorylation of ERK and Akt, which are established downstream targets of CCN1. SARI expression inhibited the phosphorylation of ERK and AKT/PI3 kinases in both MCF-pcDNA3.1 and MCF-CCN1 cl1 and MCF-CCN1 cl2. CCN1 is a secreted protein and enhances MAPK and AKT/PI3 kinases though activation of integrins αvβ3 in breast cancer cells. Over expression SARI enhanced anchorage independent growth of MCF-7 cells (FIG. 12) also their ability to invade though Matrigel. When infected with a low dose of Ad.SARI (20 pfu/cell), both anchorage independence in MCF-pcDNA3.1 and anchorage independence and invasion of MCF-CCN1 cl1 and MCF-CCN1 cl2 cells were inhibited (FIG. 12).

### Specific Example 9

### SARI Eradicates CCN1-Induced Tumors Generated by Breast Cancer Cells in Nude Mice

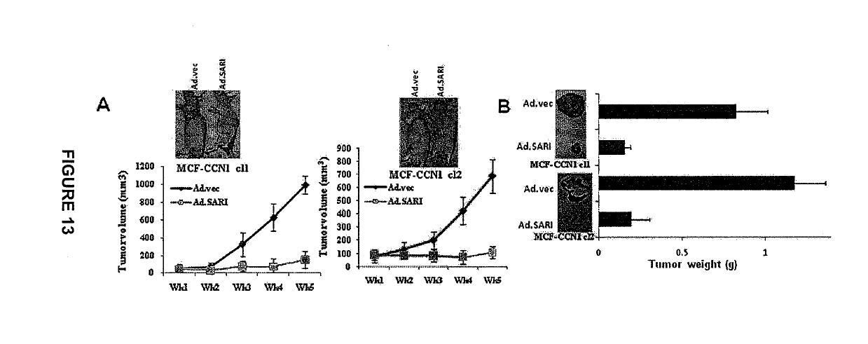

This study in this example determined whether the in vitro effects of CCN1 and SARI could be recapitulated in vivo. To achieve this objective, MCF-7, MCF-CCN1 cl1 and MCF-CCN1 cl2 tumors were established in the flanks of athymic nude mice by injecting 1×10⁶ cells/animal. The MCF-7 tumors initially grew slowly but then disappeared within a week (data not shown). In contrast, MCF-CCN1 cl1 and MCF-CCN1 cl2 cells generated actively growing tumors. After palpable tumors of −100 mm³ developed, in ˜7 to 10 days, the animals received 7 intratumoral injections over a 4-week period with 1×10⁸ pfu/injection of Ad.-vec or Ad.SARI. Both MCF-CCN1 cl1 and MCF-CCN1 cl2 cells formed large, aggressive and actively proliferating tumors in Ad.vec-infected animals, whereas Ad.SARI injections dramatically inhibited tumor growth, which was evident by wk 2 after initiating the therapeutic treatment protocol (FIGS. 13A and 13B). The efficacy of Ad.SARI for in vivo transgene delivery and therapeutic activity by immunohistochemical staining for SARI protein, Ki-67 (which measures proliferation) and CD31 (which measures angiogenesis), respectively, was also examined. In both MCF-CCN1 cl1 and MCF-CCN1 cl2 nude mice xenografts, infection with Ad.SARI resulted in significant SARI protein, that correlated with decreased expression of Ki-67 and CD-31 staining.

### Specific Example 10

### SARI Inhibits CCN1 Promoter Function by Interfering with AP-1 Activation

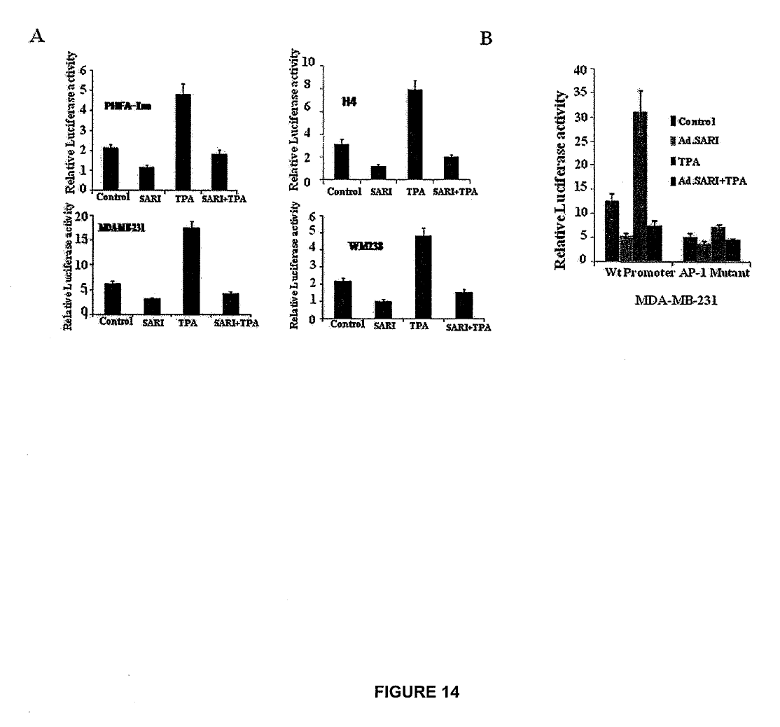

CCN1 promoter activation as a function of forced expression of SARI in breast cancer, melanoma and malignant glioma in the presence or absence of TPA (FIG. 14A) was evaluated to elucidate the mechanism by which SARI inhibits CCN1 transcription. Cells were transfected with CCN1/pGL3Luc (a full length CCN1 promoter cloned in the pGL3 basic vector) followed by infection with Ad.SARI (10 pfu/cell). SARI inhibited both control and TPA-induced CCN1 promoter function within 24 h in all three-cancer indications (FIG. 14A). This activation and inhibition was reduced (FIG. 14B) when the MDA-MB-231 cells were transfected with a CCN1(-AP-1)/pGL3Luc construct (a CCN1 promoter that lacks the AP-1 binding site). The overall luciferase activity of MDA-MB-231 cells transfected with CCN1(-AP1)/pGL3 without any treatment was lower as compared to cells transfected with CCN1/pGL3. These results indicate that maximal CCN1 promoter activity was dependent upon AP-1 activation and SARI inhibition of CCN1 promoter function that lacks the AP-1 binding site is significantly reduced.

It is assumed that SARI may inhibit CCN1 promoter function by interfering with its AP-1 binding activity. This was tested by gel shift assays in all three-cancer indications using a consensus AP-1 probe. For gel shift assays, the control (DMSO) and TPA-treated cells were infected with Ad.SARI (20 pfu/cell) and 24 h later nuclear proteins were collected and assayed by EMSA. In control (DMSO) or TPA-treated samples the level of AP-1-binding activity was markedly reduced following infection with Ad.SARI. As predicted from CCN1 mRNA and protein expression, the baseline level of AP-1 binding in immortal normal astrocytes (PHFA-Im) and weakly aggressive melanoma (WM35) and breast carcinoma cells (MDA-MB-453) was lower than in aggressive metastatic melanoma (MW238) and malignant glioma (H4) cells.

### Specific Example 11

### SARI Attenuates AP-1 Transactivation of c-JUN Binding to cis Elements in the CCN1 Promoter

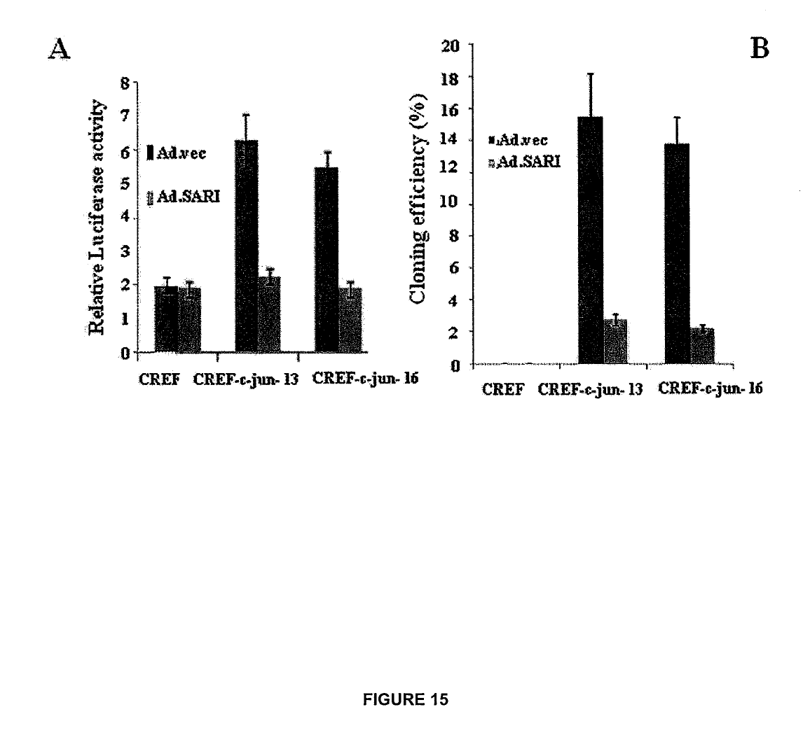

This example demonstrated that SARI down regulates CCN1 promoter function by inhibiting AP-1 binding activity. SARI interacts with c-JUN resulting in inhibition of DNA binding of AP-1 to its recognition sequence in the CCN1 promoter. To validate this assumption, CCN1 promoter activity in normal immortal cloned rat embryo fibroblast (CREF) cells, and CREF-c-jun-13 and CREF-c-jun-16 (CREF clones stably over expressing c-JUN protein) in the absence and presence of SARI (FIG. 15) was evaluated. Infection with Ad.SARI resulted in inhibition of CCN1 promoter activity in CREF-c-jun-13 and CREF-c-jun-16, but not in parental CREF cells (FIG. 15 left panel)). Similarly, infection with Ad.SARI also exerted a profound inhibitory effect in agar colony formation and invasive ability of CREF-c-jun-13 and CREF-c-jun-16 cells (FIG. 15 right panel). These effects might be a consequence of Ad.SARI down-regulating c-JUN, as well as CCN1 protein expression. Similarly, infection with Ad.SARI resulted in inhibition of AP-1 binding activity to the CCN1 promoter in both CREF-c-jun-13 and CREF-c-jun-16 cells, which was evident from EMSA assays using a consensus AP-1 probe.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An isolated nucleic acid comprising a polynucleotide encoding a polypeptide at least 95% identical to SEQ ID NO:2.

2. The isolated nucleic acid of claim 1, wherein the polypeptide comprises SEQ ID NO:2.

3. The isolated nucleic acid of claim 1, wherein the polynucleotide comprises SEQ ID NO:1.

4. An expression cassette comprising a promoter operably linked to the nucleic acid of claim 1.

5. (canceled)

6. A cell comprising the expression cassette of claim 4, wherein the expression cassette is heterologous to the cell.

7-18. (canceled)

19. A method of suppressing cancer cell growth and/or inducing apoptosis in a cancer cell, the method comprising introducing the expression cassette of claim 4 into the cell and inducing expression of the polypeptide encoded by the expression cassette.

20. The method of claim 19, wherein the method is performed in vitro.

21. The method of claim 19, wherein the method is performed in vivo.

22. The method of claim 19, wherein the cancer cell expresses CCN1 or CCN2 before the induced expression of the polypeptide.

23. The method of claim 19, wherein the cancer cell expresses AEG-1 before the induced expression of the polypeptide.

24. The method of claim 19, wherein the cancer cell is selected from the group consisting of a breast cancer cell, a glioblastoma multiforme cell, a lung cancer cell, a melanoma cell, a prostate cancer cell, a ovarian cancer cell, a cervical cancer cell, an osteosarcoma cell, a fibrosarcoma cell, and a liver cancer cell.

25-44. (canceled)


SEQUENCE LISTING (8 sequences)

SEQ ID NO: 1 - DNA, 2142 bp, Homo sapiens
  suppressor ofactivator protein-1 (AP-1), regulated by type I IFN (SARI) cDNA
gaaactgaaacttggccctctgggggcggagtggccactggggatttaaagagctgccacttccttaggcctccagagggcactgggaagtcacagctgctgagggaccactctgctcccccgcctaagccatgcacctctgtgggggcaatgggctgctgacccagacagaccccaaggagcaacaaaggcagctgaagaagcagaagaaccgggcagccgcccagcgaagccggcagaagcacacagacaaggcagacgccctgcaccagcagcacgagtctctggaaaaagacaacctcgccctgcggaaggagatccagtccctgcaggccgagctggcgtggtggagccggaccctgcacgtgcatgagcgcctgtgccccatggattgtgcctcctgctcagctccagggctcctgggctgctgggaccaggctgaggggctcctgggccctggcccacagggacaacatggctgccgggagcagctggagctgttccagaccccgggttcctgttacccagctcagccgctctctccaggtccacagcctcatgattctcccagcctcctccagtgccccctgccctcactgtcccttggccccgctgtggttgctgaacctcctgtccagctgtcccccagccctctcctgtttgcctcgcacactggttccagcctgcaggggtcttcctctaagctcagtgccctccagcccagcctcacggcccaaactgcccctccacagcccctcgagctggagcatcccaccagagggaagctggggtcctctcccgacaacccttcctctgccctggggcttgcacgtctgcagagcagggagcacaaacctgctctctcagcagccacttggcaagggctggttgtggatcccagccctcaccctctcctggcctttcctctgctctcctctgctcaagtccacttctaacctggtcttcggagctgggttggccccttctttgggctcaggaagcagccttagcacacgggcctctcctccctcactactgggtgctgccctgcgtggctgaccagctggcccaggatttcacagtcgaaaaggaagccaccactgatgcctcccactgtgacaggccctgtcaccaccaatatcttatttcaacctcacagttgacctgagaaatcgagattatcactccactttttcagacaaggaaactgaggctcagggaagccaagtgacaagtccaaggtcacgaagactttcttggagcccgaaacaccaccctctgctcctccttctcctgtcctggcccaggcatcctaggggctgaaatcctggaaaccgtgggctggtgtgagaaggtttgcatgctcagagcagagaagggctctccccactgcttcgtgattccagggccagagccatgcagtcccagaaaccccaacctagctggggcaggtccagagtccaagccctggtgggtagaggccaagcagaagccctgaagtggactcttgcttcccctagtagtgttttcagtgccaagaagctgaaactgtgagctggagttggggagaggtctggaagaggaccatctgggatttctacagcctgggtacccatagccacaccaaggcttctgggagattctgcagggtcagctttccaggctgttcccaaatagctccctgcctccccactgcccctaaagccacagcagaagagccattcatctcataaacaaaaaggaagaggaaagaatgaggaaggaccctgtgcaaggttatttgcaggcagggatgggcttgtacctgacagcacccacccctgtgtggcccccaggccctcatcaccctcagacccctcctaagcagttccctcattgctctttggactaggctgacagcaggaagagcagggcccatgaccgggtggaagttcagttttggtgtctgcttcaagagggggttttacactctgattccaggacaagcactctgaggcgggtgggggagagaaaccctggctcttcacccaggtttcacacacatgtaaatgaaacactatgttagtatctaacacactcctggatacagaacacaagtcttggcacatatgtgatggaaataaagtgttttgcaatctttaa

SEQ ID NO: 2 - protein, 274 aa, Homo sapiens
  suppressor ofactivator protein-1 (AP-1), regulated by type I IFN (SARI)
MHLCGGNGLLTQTDPKEQQRQLKKQKNRAAAQRSRQKHTDKADALHQQHESLEKDNLALRKEIQSLQAELAWWSRTLHVHERLCPMDCASCSAPGLLGCWDQAEGLLGPGPQGQHGCREQLELFQTPGSCYPAQPLSPGPQPHDSPSLLQCPLPSLSLGPAVVAEPPVQLSPSPLLFASHTGSSLQGSSSKLSALQPSLTAQTAPPQPLELEHPTRGKLGSSPDNPSSALGLARLQSREHKPALSAATWQGLVVDPSPHPLLAFPLLSSAQVHF

SEQ ID NO: 3 - RNA, 2142 nt, Homo sapiens
  suppressor ofactivator protein-1 (AP-1), regulated by type I IFN (SARI) mRNA
gaaacugaaacuuggcccucugggggcggaguggccacuggggauuuaaagagcugccacuuccuuaggccuccagagggcacugggaagucacagcugcugagggaccacucugcucccccgccuaagccaugcaccucugugggggcaaugggcugcugacccagacagaccccaaggagcaacaaaggcagcugaagaagcagaagaaccgggcagccgcccagcgaagccggcagaagcacacagacaaggcagacgcccugcaccagcagcacgagucucuggaaaaagacaaccucgcccugcggaaggagauccagucccugcaggccgagcuggcgugguggagccggacccugcacgugcaugagcgccugugccccauggauugugccuccugcucagcuccagggcuccugggcugcugggaccaggcugaggggcuccugggcccuggcccacagggacaacauggcugccgggagcagcuggagcuguuccagaccccggguuccuguuacccagcucagccgcucucuccagguccacagccucaugauucucccagccuccuccagugcccccugcccucacugucccuuggccccgcugugguugcugaaccuccuguccagcugucccccagcccucuccuguuugccucgcacacugguuccagccugcaggggucuuccucuaagcucagugcccuccagcccagccucacggcccaaacugccccuccacagccccucgagcuggagcaucccaccagagggaagcugggguccucucccgacaacccuuccucugcccuggggcuugcacgucugcagagcagggagcacaaaccugcucucucagcagccacuuggcaagggcugguuguggaucccagcccucacccucuccuggccuuuccucugcucuccucugcucaaguccacuucuaaccuggucuucggagcuggguuggccccuucuuugggcucaggaagcagccuuagcacacgggccucuccucccucacuacugggugcugcccugcguggcugaccagcuggcccaggauuucacagucgaaaaggaagccaccacugaugccucccacugugacaggcccugucaccaccaauaucuuauuucaaccucacaguugaccugagaaaucgagauuaucacuccacuuuuucagacaaggaaacugaggcucagggaagccaagugacaaguccaaggucacgaagacuuucuuggagcccgaaacaccacccucugcuccuccuucuccuguccuggcccaggcauccuaggggcugaaauccuggaaaccgugggcuggugugagaagguuugcaugcucagagcagagaagggcucuccccacugcuucgugauuccagggccagagccaugcagucccagaaaccccaaccuagcuggggcagguccagaguccaagcccugguggguagaggccaagcagaagcccugaaguggacucuugcuuccccuaguaguguuuucagugccaagaagcugaaacugugagcuggaguuggggagaggucuggaagaggaccaucugggauuucuacagccuggguacccauagccacaccaaggcuucugggagauucugcagggucagcuuuccaggcuguucccaaauagcucccugccuccccacugccccuaaagccacagcagaagagccauucaucucauaaacaaaaaggaagaggaaagaaugaggaaggacccugugcaagguuauuugcaggcagggaugggcuuguaccugacagcacccaccccuguguggcccccaggcccucaucacccucagaccccuccuaagcaguucccucauugcucuuuggacuaggcugacagcaggaagagcagggcccaugaccggguggaaguucaguuuuggugucugcuucaagaggggguuuuacacucugauuccaggacaagcacucugaggcgggugggggagagaaacccuggcucuucacccagguuucacacacauguaaaugaaacacuauguuaguaucuaacacacuccuggauacagaacacaagucuuggcacauaugugauggaaauaaaguguuuugcaaucuuuaa

SEQ ID NO: 4 - DNA, 1478 bp, Homo sapiens
  promoter (1)...(1478): suppressor of activator protein-1 (AP-1), regulated by type I IFN (SARI) promoter
tccatgaactctttaaaacaacattccagccaccatcacctatcagaagatcatatctgtatttgccattcttgtgataccacatcatcagccatgagccctcaggtctcaggtacaactccaagatgctctagtcaccagaccataggactctacatactgcatgcatgctgttcttggacaggaattactggcccctctggcccccaaacagatgccccatccaagtgtccctcttagagatacaacaagcatgcaggtatgcccggggcttcagggtttgctaggtctttattttttaatttttaattaattaatttttttgaaacaaggtcttgctctattgcccaggctggagtgcagtggcaagattatagctcactgcagccttgaactcctgggctcaagtgatcctctcatctcagcctcctgagtaactgggaatacaggtgtgtgccaccatgcccagttattttttttatttttaatatagataaggtcttgctatgttgcccaggctggtctcaaactcctgagctcaagtaatcctcctgccttggcctcccagagtacttggattacaggcatgaaccactgcatctggcctaacaaaattgtttttatccccactttacagatttggaaacagaaacacagatgaaattacgaacccaagtcacatagcttggaagctgggctcctaaccactggacatcaatctttccactgcttcatggtctgttgtatgaaaacagtaatgtatccatctgttctatccaacagccattgcttaattttgcagacatgcttccatagtggtctcagaaggtttgggggtggttgtattatctggcttagttcctgcaatgagcctgggagtgggtagttgtaccatcctccagatgagaaaactagggctcagagaaggagagtcactttctgaaagttggacagctccaatgtgtgctgtgatccagagttcccccaattccagagctcaagggctctctctaccccacatgagcatcggtccccactggcttcttctctcatctcagtccctggccgcctgtcctccctcccctagaacaccctcctcatacctggcctttggtcttccctcctctccaccctccaccatcctctttccagagaaggggcccaaccggaaggccagttcatgttaggaaattctagccacgtgagcaagcctgagccagggattctgagcagctggtggctttaggagaaactgaaacttggccctctgggggcggagtggccactggggatttaaagagctgccacttccttaggcctccagagggcactgggaagtcacagctgctgagggaccactctgctcccccgcctaagccatgcacctctgtgggggcaatgggctgctgacccagacagaccccaaggagcaacaaaggcagctgaagaagcagaagaaccgggcagccgcccagcga

SEQ ID NO: 5 - DNA, 951 bp, Homo sapiens
  promoter (1)...(951): secretory integrin-binding protein CCN1 cancer-specific promoter
tccaaaaacaaacaagtacaacatacccaaaagagggaagggctggaggagtgggggagacctctgcctgggaatttgccagacgatgggcaagtttccccccgccccaccccccccccgccttttcattcataaatgccactgtgggtattaatttgcaattcactgaactttgctaataaacatcatgccaaagctttgggacttgttccgaacacgcctctttgaagtccacaaatattcctgactcagagacacactcctcttccccgttctactctttcaacagataacttgcctctcaccttcgctgtaaaaaagcaaacagctcactgccttcccgggtgagggcttcagtggctgcccggtcaactcgcatcaccaaacaaaacgacttttgttcctccctctcaggtcctcccacccacccagtccaggcaaagttctgaactggcccccctcgcccctcacgaccctccaactaccatcaccaccatcacgccccaaagaacccttcccaacataagtcgtaatttaaggtggaaaaaacgaactgttttcttgacgggtctgggacacacacacacacacacacacacacacacacaccccccgaactgttttcttgacgggtctgggagacagacacacacacagacacacacacacacacacacacacacacacacacacacacacacacacacacacaaaggtgcaatggagccaggggaggcgcttggcagcagcccgcgccaaccagcattcctgagatgtttgagaattctggaacgcgcagacagagccgacgtcactgcaacacgcggcgcctccgccggcccgtataaaaggcgggctccggggcgcctgggcagaccgcgagcgagagcgcccccgagcagcgcccgcgccctccgcgccttctccgccgggacctcgagcgaaagacgc

SEQ ID NO: 6 - DNA, 1940 bp, Homo sapiens
  promoter (1)...(1940): PEG-3 cancer-specific promoter
tggctgggcaacacgggttcagcccaggtttcatagtaagttccagacactcctggaaaaacaatacaggtccctgacaaaagaaaaaacaaaacaaaggaaacagaaacatgcgtttttaaaaaagaaggaggagactccatgaaggcaggccttgggtggggtcactgcttctctgtacacaggaggagaattgccaagatcttccggacagtgtggactatactgtaagaccctctcaatacagacagactggacaggcatagtgacacatgcctttaatgcctgcagtactcaggaggaggtggcaggtggaacggctgttctttgaggttcaagaccagcgtggactacagagtgagttccaggacaggcagggctacacagaaaaatcctgtctgaaaacaaaacaaaacccagacagacacaccaaaaacagccaagggaccagagagatgggtcagggcctaatcacttgctactctttgcagaggacccaaatttagttcctataaccctccatgagaagcttcacaattgtctctaactcaattccacccgtgttccgacctcccatatgcaccagacatgttatactcacacatacgcacaaacacacacacacacacacacacacacacacacacacacacacacacacggaaaacatataaaataaagatttaaaaaatctttttcttttggccggggtgtgtgggagagcatctgagccatctcaccagcccagggtgcacgtctttttctttttttcggagctggggaccgaacccagagccttgtgcttgctaggcaagtgctctaccactgagctaaatccccaaccccggagcacgtctttaatcccagaatcaggaggtagaggtaatgagatcccagtgagcccaaggtcagccgagtctacaaagtgagttccaggacagccagaactaatcttggaaaaacaaacaagggctggtgaggtggttcagtagttaagaacactggctgctcttccagaggtcctgagttcattctcagtaaccacatggtggggatctgatgcctgttctggcatgcagatatacatgcagatagtgcactcctacatttaaaaaaaaaagacataaataatattttaaaacattgggcgttttgtcttctaataaaacttcactgctatcttctaataaaaattcactgctagccgcggggtgtggtgcccccatacctttaatcccaacaacttgagaggcagaggcaggcggacctttgagtttgaagctagcctggtctacagagtgagttcaagatagccacggatagtcagaaagtcctgtttcgaacctctccccaaccaaatcactcctgtaatcccagcactctggaggcagtagcaggttagtccctgcttctcagagagaggagagagagagagagagagaggagacacacacacacagagacagagaggagagagaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 7 - DNA, 1975 bp, Homo sapiens
  tumor-suppressor gene mda-7/IL-24 cDNA
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtcttctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactcaagtctggcagactttcttctctatttctggatgaatgcccagtgagactgtgttgtacagctagaaaaggccttcttcccaatagcaaggctgtgcatctagcctcaagctctggctgaactttgtggtcgacatcaatctaaagatacagtgtctgactataaccttgttccaaaaacctaggcaaagagtatatgtaggaggtgggatatcacttccatgacataagtgctattgcagagccgtggccacccaggaactcctgactgctttcc

SEQ ID NO: 8 - protein, 22 aa, Artificial Sequence
  synthetic SARI protein basic-region leucine zipper motif
LXXXXXXLXXXXXXLXXXXXXL